(19) United States
(12) Patent Application Publication
(10) Pub. No.: US 2004/0175822 A1
(43) Pub. Date: Sep. 9, 2004

(54) Apparatus and method for edman degradation using a microfluidic system

(75) Inventor: Aaron T. Timperman, Morgantown, WV (US)

Correspondence Address: PALMER & DODGE, LLP, PAULA CAMPBELL EVANS, 111 HUNTINGTON AVENUE, BOSTON, MA, 02199, US

(73) Assignee: West Virginia University Research Corporation

(21) Appl. No.: 10/740,188

(22) Filed: Dec. 18, 2003

Related U.S. Application Data
(60) Provisional application No. 60/434,746, filed on Dec. 18, 2002.

(51) Int. Cl. C12M 1/34; G01N 33/553

(52) U.S. Cl. 435/287.2; 436/526

(57) ABSTRACT

An apparatus and method for Edman degradation using a microfluidic system to identify and characterize peptides is disclosed. A microfluidic device comprises an entrance channel through which a substantially purified polypeptide is accepted, a reaction channel engaging the entrance channel wherein the substantially purified polypeptide is digested, producing a digestion product, a reagent reservoir engaging the reaction channel, the reagent reservoir capable of delivering a reagent to the reaction channel, and an exit channel extending from the reaction channel, wherein the digestion product travels through the exit channel upon leaving the reaction channel. Protein digestion on the device comprises delivering a substantially purified polypeptide to a reaction channel, confining the polypeptide in the reaction channel, digesting the polypeptide in the reaction channel producing a digestion product, and removing the digestion product from the reaction channel, wherein the last two steps are repeated until the polypeptide is substantially digested.

### RELATED APPLICATIONS

This application claims priority from U.S. Provisional Patent Application Serial No. 60/434,746, filed on Dec. 18, 2002 the entirety of which is hereby incorporated by reference.

### FIELD OF THE INVENTION

The present invention relates to a microfluidic system, and, more particularly, to an apparatus and method for performing Edman degradation on a microfluidic device.

### BACKGROUND OF THE INVENTION

The goal of proteomics is to identify and quantitate all of the proteins expressed in a cell as a means of addressing the complexity of biological systems (Anderson, 1998, Electrophoresis 19: 1853-1861). Current methods for proteome analysis generally are based on the use of two-dimensional electrophoresis (2DE) to identify cellular proteins. Protein patterns on 2DE gels are analyzed using image analysis techniques to generate proteome maps. Proteome maps of normal cells and diseased cells are compared to detect proteins that are up- or down-regulated during physiological responses to disease. These proteins are excised for identification and characterization, using such methods as mass fingerprinting and mass spectrometry.

However, using current 2DE methods, only the most abundant proteins can be identified. Thus, most of the proteins identified by 2DE methods represent structural proteins or housekeeping proteins (see, e.g., Gygi et al., 2000, Proc. Natl. Acad. Sci. USA 97: 9390-9395; Gygi et al., 1999, Electrophoresis 20: 310-319; Shevchenko, 1996, Proc. Nat. Acad. Sci. USA 93: 14440-14445; Boucherie, 1996, Electrophoresis 17(11): 1683-1699; Ducret, 1998, Protein Science 7: 706-719; Garrels, 1994, Electrophoresis 15: 1466-1486). These problems have limited the use of proteomics for the identification of cancer markers because the lower abundance proteins that produce aberrant cell signals cannot be qualified, making it difficult to elucidate mechanisms that cause disease states and identify suitable cancer-specific markers.

The lack of sensitivity of current 2DE-based technology is caused primarily by a lack of separating or resolving power because high abundance proteins mask the identification of low abundance proteins. Loading more protein on the gels does not improve the situation because the Gaussian tails of the high abundance spots contaminate the low abundance proteins. The use of zoom gels (2D gels that focus on a narrow pH range) allows for minimal gains (Gygi, 2000, supra) but is considered too cumbersome to be of any practical utility (Corthals, 2000, Electrophoresis 21: 1104-1115). Selective enrichment methods also can be used but generally at the expense of obtaining a comprehensive view of cellular protein expression. The sensitivity of detection on 2DE gels also is problematic, because the amount of protein required for identification by mass spectrometry (MS) is near the detection limits of the most sensitive methods for visualization of the protein spots on the 2DE gels. Further, the polyacrylamide matrix typically used in 2DE gives rise to a significant amount of background in the extracted sample mixture making subsequent analysis by MS difficult (Kinter, 2000, In Protein Sequencing and Identification Using Tandem Mass Spectrometry, Wiley, N.Y.). Additionally, during peptide extraction following typical in-gel digestion procedures, the sample is exposed to many surfaces and losses can be substantial, particularly for low abundance proteins (Timperman, 2000, Anal. Chem. 72: 4115-4121; Kinter, supra).

Multi-dimensional column separations offer many advantages over 2DE, including a higher separating power and reduced sample contamination and loss. A typical large format 2DE gel is capable of achieving a peak capacity of about 2,000 while 2D column separations can achieve peak capacities of over 20,000 for protein separations. Additionally, the stationary phases of these columns are very stable and non-reactive compared to polyacrylamide gels, leading to reduced sample contamination and loss. Many different types of separation techniques have been coupled to 2D column separations including size exclusion, reversed phase chromatography, cation-exchange chromatography, and capillary electrophoresis (Wall, 2000, Analytical Chemistry 72: 1099-1111; Link, 1999, Nature Biotechnology 17: 676-682; Opiteck, 1998, Journal of Microcolumn Separations 10: 365-375; Hooker et al., 1998, In High-Performance Capillary Electrophoresis, John Wiley & Sons Inc, New York, Vol. 146, pp 581-612; Opiteck et al., 1998, Analytical Biochemistry 258: 349-361; Vissers, 1999, Journal of Microcolumn Separations 11: 277-286.; Liu et al., 1996, Anal. Chem. 68: 3928-3933.) Further increases in peak capacity have been achieved using three-dimensional columns (see, e.g., Moore, 1995, supra).

Edman degradation commonly has been used for sequencing proteins and peptides since its introduction by Pehr Edman. The Edman degradation method for peptide and protein sequencing is based on the cyclic removal and identification of the terminal amino acid. Edman degradation is based on a labeling reaction between the terminal amino group and phenyl isothiocyanate, C₆H₅N═C═S. When the labeled polypeptide is treated with acid, the terminal amino acid residue is cleaved as an unstable intermediate that undergoes rearrangement to a phenylthiohydantoin. This last product can be identified by comparison with phenylthiohydantoin prepared from standard amino acids. The sequence of the protein or polypeptide is elucidated by cycling the protein or peptide through many stages of removal and sequential identification of the terminal amino acid residue. The original Edman degradation chemistry was based on coupling phenylisothiocyanate (PITC) to the N-terminal amino group. Since the original development with PITC, many other analogues to PITC have been developed which offer improved reactivity or detection. In addition, chemistries have been developed for coupling and sequencing from the C-terminal carboxy group.

Recently, Edman sequencers have been miniaturized. A gas phase membrane sequencer was developed by Wurzel et al. However, this gas phase sequencer is not as easy to couple with liquid separations as a solid phase sequencer. Also, the gas phase membrane sequencer requires relatively large volumes. (Wurzel, C. W.-L., Brigitte (2000) Proteomics in Functional Genomics 88: 145-157); (Wurzel, C. W.-L., Brigitte (1998) Springer, Berlin, Germany: 219-224); (Wurzel, C. W.-L., B (1998) Journal of Protein Chemistry 17(6): 561-564).

Microfluidic devices are finding many applications for DNA analysis, but there has been little development in the utilization of these devices in the field of protein analysis. The microfluidic device revolution was begun by Harrison, 1992, Analytical Chemistry 64: 1926-1932, who demonstrated valveless electrophoretic separation and fluid manipulation on such microfluidic devices. Much recent work has focused on the basics of sample injection, on-device column fabrication and interfacing with mass spectrometry. Harrison et al. have developed a microfluidic device that incorporates on chip digestion of proteins using a proteolytic enzyme. The device has been designed for high flow to allow for the concentration and identification of proteins from gel spots. In the prior art, a microfluidic device has been disclosed for fractionating microliter volumes of peptides from a protein digest.

Capillary systems for performing proteolytic digestions (see, e.g., Licklider et al., 1995, Analytical Chemistry 67: 4170-4177; Licklider et al., 1998, Analytical Chemistry 70: 1902-1908) and microfluidic devices for protease digestion have been described (see, e.g., Tremblay et al., 2001, Proteomics 1(8): 975-986; Li et al., 2001, Eur. J. Mass Spectrom. 7(2): 143-155; Li et al., 1999, Anal. Chem. 71: 3036-3045; Khandurina et al., Anal. Chem. 71: 1815-1819).

Edman degradation has not been used extensively for sequencing gel separated proteins due to its poor sensitivity and difficulties involved with fractionating digested proteins into their resultant peptides at pmol to fmol levels. Therefore, a need remains in the art for a microfluidic device capable of utilizing Edman degradation for protein sequencing.

### SUMMARY OF THE INVENTION

The present invention comprises an apparatus and method for Edman degradation using a microfluidic system to identify and characterize a peptide or a polypeptide. Edman degradation comprises utililyzing a stepwise cleavage and identification of a terminal amino acid of a peptide or a polypeptide. The present invention provides an apparatus and method of collecting a substantially purified polypeptide on a microfluidic device, cleaving the terminal amino acid, and separating the cleaved amino acid from the microfluidic device. The present invention further comprises an apparatus and method of identifying the cleaved amino acid and thereby determining the amino acid sequence of the substantially purified polypeptide.

In one embodiment of the present invention, the apparatus comprises a microfluidic device comprising a substrate defining an entrance channel through which a substantially purified polypeptide can be accepted into a reaction channel of the microfluidic device. The reaction channel is in fluid communication with a plurality of reagent reservoirs, the reagent reservoirs capable of delivering a plurality of reagents to the reaction channel. Once inside the reaction channel, the substantially purified polypeptide is subjected to Edman degradation, thereby producing a cleavage product. The device of the present invention further defines an exit channel, wherein the cleavage product travels through the exit channel upon leaving the reaction channel. The device preferably comprises a planar substrate or chip, or a three-dimensional network.

In one embodiment of the present invention, at least one reaction channel includes a first solid phase for engaging the substantially purified polypeptide in preparation for the cleavage reaction. In one embodiment of the present invention, the reaction channel can comprise particles or beads. The particles or beads may include one or more reagents immobilized thereon. In one embodiment of the invention, the first solid phase comprises a plurality of beads for immobilization of the peptide or polypeptide on the plurality of beads. In an embodiment of the present invention, a plurality of beads are used to confine the substantially purified polypeptide to the reaction channel. In one embodiment, an at least one membrane can be used to confine the substantially purified polypeptide to the reaction channel.

In a preferred embodiment of the present invention, the microfluidic device is a chip comprising a plurality of reaction channels and Edman degradation is performed on a substantially purified polypeptide in each of the plurality of reaction channels. Reagents for performing the Edman reactions can be stored in a plurality of reagent reservoirs, and transported as needed to the reaction channel(s).

The present invention also provides a method for performing Edman degradation on a microfluidic device. The method of the present invention comprises delivering a substantially purified polypeptide to a reaction channel of the microfluidic device. In one embodiment, the substantially purified polypeptide is confined to the reaction channel, and while in the reaction channel, can be contacted with a plurality of reagents to perform chemical reactions on the substantially purified polypeptide. The reagents access the reaction channel via the plurality of reagent reservoirs. In a preferred embodiment of the present invention, Edman degradation is used to cleave the terminal amino acid of the substantially purified polypeptide in the reaction channel. In this embodiment, the cleavage product is an amino acid cleaved from the N-terminal end of the substantially purified polypeptide. In another preferred embodiment, the cleavage product is a small peptide or an amino acid cleaved from the C-terminal end of the substantially purified polypeptide.

In a preferred embodiment of the present invention, the cleavage product, a cleaved amino acid, is concentrated in the reaction channel. This concentrated small peptide or amino acid can then be removed from the reaction channel. In a preferred embodiment of the present invention, the cleavage product is removed from the reaction channel and sent to a detector. In a preferred embodiment, the cleaved amino acid is removed from the reaction channel and identified before the next cleavage reaction takes place. In a preferred embodiment, the steps of cleaving the terminal amino acid, concentrating the cleaved amino acid, removing the concentrated cleaved amino acid from the reaction channel and detecting and/or identifying the concentrated amino acid are repeated until each amino acid of the substantially purified polypeptide has been identified or until the detection limit has been reached.

The microfluidic device and the various channels defined thereby may comprise varying geometries. In a preferred embodiment, the microfluidic device is a substantially planar substrate (“chip”) defining an entrance channel which divides into a plurality of substantially parallel sample reaction channels, which then converge again at an exit channel. It is not necessary that these channels be geometrically parallel, but preferably, they are configured as a set of parallel openings having a common entrance channel and a common exit channel.

In another embodiment of the present invention, the microfluidic device comprises substantially parallel channels which are intersected by substantially perpendicular channels. The channel geometry is not critical so long as the appropriate fluid flow relationships are maintained. For example, channels can be curved or angled, or the substrate itself can be not planar and the channels can be non-coplanar.

The microfluidic device can be substantially covered with an overlying substrate to form the channels. In an embodiment of the present invention, the overlying substrate defines at least one opening for communicating with a least one channel in the microfluidic device. Openings can be used to add reagents, fluids, or other materials to the microfluidic device.

The present invention further provides an integrated microfluidic proteomic analysis system as disclosed in Applicants' pending application Serial No. 60/344,456 wherein the system further comprises a microfluidic device capable of Edman degradation. The system includes a first microfluidic device for utililyzing Edman degradation. The microfluidic system further comprises an upstream separation module capable of separating a plurality of polypeptides or proteins. The upstream separation module delivers a substantially purified polypeptide to an at least one entrance channel of the microfluidic device. In one embodiment, the upstream separation module separates a sample comprising a plurality of polypeptides according to at least a first and a second criteria, wherein the first and second criteria are different. For example, the first criteria may be molecular mass and the second criteria may be isoelectric point or vice versa. In a preferred embodiment, the upstream separation module comprises a first separation path for separating the sample comprising the plurality of polypeptides according to the first criteria, and a second separation path for separating polypeptides which have been separated according to the first criteria, according to the second criteria. In one embodiment, the protein or polypeptide is added directly to the microfluidic device capable of Edman degradation and bypasses the upstream separation module.

The exit channel of the microfluidic device may be connected to a detection system for identification of the cleaved amino acid. The microfluidic device of the present invention is in fluid communication with a downstream separation module for separating cleavage products of the cleavage reaction. In one embodiment of the present invention, the downstream separation module is in fluid communication with a peptide analysis module (e.g., such as a mass spectrometer) for determining one or more ionization properties of the cleavage products. The peptide analysis module may comprise an electrospray ionization mass spectrometer (ESI MS) instrument. In another embodiment, the downstream separation module is omitted, and the peptide analysis module is directly connected with the microfluidic device. The system may comprise a plurality of peptide analysis modules.

In one embodiment of the present invention, the peptide analysis module is in communication with a detector for identifying the cleavage products. The identification of the cleaved amino acid can then be used to determine the sequence of the substantially purified polypeptide. In a preferred embodiment, information relating to the amino acid sequence of the substantially purified polypeptide is stored in a database.

In a preferred embodiment of the present invention, the microfluidic device is in electrical communication with one or more electrodes connectable to a power source for selectively applying a voltage at one or more of the various channels or reservoirs of the microfluidic device. The voltage can be used to drive the transport of reagents, polypeptides and cleavage products through various channels of the microfluidic device. With electrical control, electroosmotic and electrophoretic transport are used for reagent and solution transport.

In another embodiment, hydrodynamic flow can be used for transport by the application of positive or negative pressures to the device. The use of microfluidic valves would allow flow to be directed in the channel. An example of such valves are hydrophobic microfluidics that use restrictions which act as passive valves.

In one embodiment, flow can be generated by centrifugal force or the combination of electric and magnetic fields to effect reagent and solution flow and delivery.

In one embodiment, an electroosmotic flow (“EOF”) pump regulates flow on the microfluidic device.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a schematic view of a preferred microfluidic system of the present invention.

FIG. 2 is a schematic view of alternative embodiments of an integrated proteomic microfluidic analysis module including a microfluidic device for Edman degradation of the present invention.

FIG. 3 is a box diagram showing the microfluidic device of the present invention.

FIG. 4 is a view of an embodiment of the microfluidic device of the present invention wherein a substantially purified polypeptide and a cleaved amino acid are concentrated in front of an ultrafiltration membrane.

FIG. 5 is a view of the embodiment as shown in FIG. 4 further comprising an electroosmotic flow pump.

FIG. 6 is a view of the embodiment as shown in FIG. 5 further comprising a plurality of hydrodynamic flow restrictors.

FIG. 7 is a preferred embodiment of the microfluidic device of the present invention wherein a substantially purified polypeptide is confined to a reaction channel by an ultrafiltration membrane.

FIG. 8 is a view of the embodiment as shown in FIG. 6 further comprising an electroosmotic flow pump.

FIG. 9 is a view of another embodiment of the microfluidic device of the present invention wherein a substantially purified polypeptide and a cleaved amino acid are concentrated in front of an ultrafiltration membrane.

FIG. 10 is a view of another embodiment of the microfluidic device of the present invention wherein a substantially purified polypeptide is immobilized on a plurality of beads and confined in a reaction channel by an ultrafiltration membrane. The cleavage products are allowed to pass through the ultrafiltration membrane.

FIG. 11 is a view of another embodiment of the microfluidic device of the present invention wherein a substantially purified polypeptide is confined to a reaction channel by an ultrafiltration membrane. The cleavage products are not allowed to pass through the ultrafiltration membrane.

FIG. 12 is a view of another embodiment of the microfluidic device of the present invention wherein a substantially purified polypeptide is confined to a reaction channel by an ultrafiltration membrane. The cleavage product is concentrated in front of a second ultrafiltration membrane before being removed from the reaction channel.

FIG. 13 is a view of an embodiment of the microfluidic device of the present invention wherein the digestion product is not concentrated before being removed from the reaction channel.

FIG. 14 is a view of an embodiment of the microfluidic device of the present invention wherein a cleaved amino acid is concentrated on a solid phase extraction device before being sent to a downstream separation module.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and sprit of the principles of the present invention.

### DETAILED DESCRIPTION

The present invention comprises an apparatus and method for identifying and characterizing a peptide or a polypeptide using Edman degradation carried out on a microfluidic device. Edman degradation comprises utililyzing a stepwise cleavage and identification of a cleavage product. The cleavage product comprises a terminal amino acid of the peptide or polypeptide. The present invention provides an apparatus and method of collecting a substantially purified polypeptide on a microfluidic device, cleaving the terminal amino acid, and separating the cleaved amino acid from the microfluidic device. The present invention further comprises an apparatus and method of identifying the cleaved amino acid and thereby determining the amino acid sequence of the peptide or polypeptide.

In one embodiment of the present invention, the apparatus comprises a microfluidic device comprising a substrate defining an entrance channel through which a substantially purified polypeptide can be accepted into a reaction channel of the microfluidic device. The reaction channel is in fluid communication with a plurality of reagent reservoirs, the reagent reservoirs capable of delivering a plurality of reagents to the reaction channel. Once inside the reaction channel, the substantially purified polypeptide is subjected to Edman degradation, thereby producing a cleavage product. The device of the present invention further defines an exit channel, wherein the cleavage product travels through the exit channel upon leaving the reaction channel. The device preferably comprises a planar substrate or chip, or a three-dimensional network.

The present invention also provides a method for performing Edman degradation on a microfluidic device. The method of the present invention comprises delivering a substantially purified polypeptide to a reaction channel of the microfluidic device. In one embodiment, the substantially purified polypeptide is confined to the reaction channel, and while in the reaction channel, can be contacted with a plurality of reagents to perform chemical reactions on the substantially purified polypeptide. The reagents access the reaction channel via the plurality of reagent reservoirs. In an embodiment of the present invention, a plurality of beads are used to confine the substantially purified polypeptide to the reaction channel. In one embodiment, an at least one membrane can be used to confine the substantially purified polypeptide to the reaction channel.

The present invention further provides an integrated microfluidic proteomic analysis system including a microfluidic device for utililyzing Edman degradation. The microfluidic apparatus for proteomic analysis further comprises an upstream separation module capable of separating a plurality of polypeptides or proteins. The upstream separation module delivers a substantially purified polypeptide to an at least one entrance channel of the microfluidic device. In one embodiment, the upstream separation module separates a sample comprising a plurality of polypeptides according to at least a first and a second criteria, wherein the first and second criteria are different. For example, the first criteria may be molecular mass and the second criteria may be isoelectric point or vice versa. In a preferred embodiment, the upstream separation module comprises a first separation path for separating the sample comprising the plurality of polypeptides according to the first criteria, and a second separation path for separating polypeptides which have been separated according to the first criteria, according to the second criteria. In one embodiment, the protein or polypeptide is added directly to the Edman degradation device and bypasses the upstream separation module.

### Definitions

The following definitions are provided for specific terms which are used in the following written description and claims.

As used herein, a “substantially purified polypeptide” refers to a polypeptide sample which comprises polypeptides of substantially the same molecular mass (e.g., greater than about 90%, preferably greater than about 95%, greater than about 98%, and up to about 100% of the polypeptides in the sample are of substantially the same molecular mass). Substantially purified polypeptides do not necessarily comprise identical polypeptide sequences.

As used herein, a “cleavage reaction” refers to a reaction within a reaction channel of the microfluidic device of the present invention in which a terminal amino acid is cleaved from an end of a peptide or polypeptide confined in the reaction channel. The cleavage reaction produces a cleavage product.

As used herein, a “cleavage product” refers to the product of the cleavage reaction. The cleavage product comprises a terminal amino acid or a small peptide cleaved from a peptide or polypeptide wherein the peptide or polypeptide is confined to the reaction channel.

As used herein, “a sample band” or “sample plug” refers to a volume of a fluid which comprises a sample (e.g., a substantially purified polypeptide or substantially purified peptide).

As used herein, a first solid phase which is “substantially adjacent” to a second solid phase in a channel describes a first solid phase in which at least a portion of the first solid phase contacts the second solid phase.

As used herein, the term, “in communication with” refers to the ability of a system or component of a system to receive input from another system or component of a system and to provide an output in response to the input. “Input” or “Output” may be in the form of electrical signals, light, data (e.g., spectral data), materials, or may be in the form of an action taken by the system or component of the system. The term “in communication with” also encompasses a physical connection which may be direct or indirect between one system and another or one component of a system and another.

As used herein, a “molecular probe” is any detectable molecule, or is a molecule which produces a detectable molecule upon reacting with a biological molecule (e.g., polypeptide or nucleic acid).

As used herein, “expression” refers to a level, form, or localization of product. For example, “expression of a protein” refers to one or more of the level, form (e.g., presence, absence or quantity of modifications, or cleavage or other processed products), or localization of the protein.

As used herein, “a diagnostic trait” is an identifying characteristic, or set of characteristics, which in totality, are diagnostic. The term “trait” encompasses both biological characteristics and experiences (e.g., exposure to a drug, occupation, place of residence). In an embodiment, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in that trait which is statistically significant within 95% confidence levels.

As used herein, a “difference in expression” or “differential expression” refers to an increase or decrease in expression compared to a norm. A difference may be an increase or a decrease in a quantitative measure (e.g., amount of a polypeptide or RNA encoding the polypeptide) or a change in a qualitative measure (e.g., a change in the localization of a polypeptide). Where a difference is observed in a quantitative measure, the difference according to the invention will be at least about 10% greater or less than the level in a normal standard sample. Where a difference is an increase, the increase may be as much as about 20%, 30%, 50%, 70%, 90%, 100% (2-fold) or more, up to and including about 5-fold, 10-fold, 20-fold, 50-fold or more. Where a difference is a decrease, the decrease may be as much as about 20%, 30%, 50%, 70%, 90%, 95%, 98%, 99% or even up to and including 100% (no specific polypeptide or RNA present). It should be noted that even qualitative differences may be represented in quantitative terms if desired. For example, a change in the intracellular localization of a polypeptide may be represented as a change in the percentage of cells showing the original localization.

As used herein a “a sample” refers to polypeptides and/or peptides. A sample can be obtained from a variety of sources including, but not limited to: a biological fluid, suspension, buffer, collection of cells, scraping, fragment or slice of tissue, a tumor, an organism (e.g., a microorganism such as a bacteria or yeast). A sample also can comprise a subcellular fraction, e.g., comprising organelles such as nuclei or mitochondria.

As used herein, a “biological fluid” includes without limitation blood, plasma, serum, sputum, urine, cerebrospinal fluid, lavages, and leukapheresis samples.

As defined herein, a “configuration of parallel channels” is one which provides a common voltage output at an intersection point between the channels. However, the geometric arrangement of the channels is not necessarily parallel. However, they should be configured as a set of parallel resistors in a circuit having a common input channel and a common output channel.

As used herein, a channel which has a geometric configuration which is “substantially parallel” to another is a channel which is at a less than 5 degree angle with respect to the longitudinal axis of the other channel. A channel which is “substantially perpendicular” another is a channel which is at a 90° angle with respect to the longitudinal axis of another channel, ±50.

As used herein, an amino acid sequence which is “assembled” from a plurality of sequences refers to an end-end connection and/or to the connection of overlapping sequences at regions of overlap.

As used herein, “a system processor” refers to a apparatus comprising a memory, a central processing unit capable of running multiple programs simultaneously, and preferably, a network connection terminal capable of sending and receiving electrical signals from at least one non-system apparatus to the terminal. The system processor is in communication with one or more system components (e.g., modules, detectors, computer workstations and the like) which in turn may have their own processors or microprocessors. These latter types of processors/microprocessors generally comprise memory and stored programs which are dedicated to a particular function (e.g., detection of fluorescent signals in the case of a detector processor, or obtaining ionization spectra in the case of a peptide analysis module processor, or controlling voltage and current settings of selected channels on a device in the case of a power supply connected to one or more devices) and are generally not directly connectable to the network. In contrast, the system processor integrates the function of processors/microprocessors associated with various system components to perform proteome analysis as described further below.

As used herein, a “database” is a collection of information or facts organized according to a data model which determines whether the data is ordered using linked files, hierarchically, according to relational tables, or according to some other model determined by the system operator. Data in the database are stored in a format consistent with an interpretation based on definitions established by the system operator.

As used herein, “a system operator” is an individual who controls access to the database.

As used herein, an “information management system” refers to a program, or series of programs, which can search a database and determine relationships between data identified as a result of such a search.

As used herein, an “interface on the display of a user apparatus” or “user interface” or “graphical user interface” is a display (comprising text and/or graphical information) displayed by the screen or monitor of a user apparatus connectable to the network which enables a user to interact with the database and information management system according to the invention.

As used herein, the term “link” refers to a point-and-click mechanism implemented on a user apparatus connectable to the network which allows a viewer to link (or jump) from one display or interface where information is referred to (“a link source”), to other screen displays where more information exists (a “link destination”). The term “link” encompasses both the display element that indicates that the information is available and a program which finds the information (e.g., within the database) and displays it on the destination screen. In an embodiment, a link is associated with text; however, in other aspects, links are associated with images or icons. In some aspects, selecting a link (e.g., by right clicking using a mouse) will cause a drop down menu to be displayed which provides a user with the option of viewing one of several interfaces. Links can also be provided in the form of action buttons, radio buttons, check buttons and the like.

As used “providing access to at least a portion of a database” refers to making information in the database available to user(s) through a visual or auditory means of communication.

As used herein, “pathway molecules” or “pathway biomolecules” are molecules involved in the same pathway and whose accumulation and/or activity and/or form (i.e., referred to collectively as the “expression” of a molecule) is dependent on other pathway molecules, or whose accumulation and/or activity and/or form affects the accumulation and/or activity or form of other pathway target molecules. For example, a “GPCR pathway molecule” is a molecule whose expression is affected by the interaction of a GPCR and its cognate ligand (a ligand which specifically binds to a GPCR and which triggers a signaling response, such as a rise in intracellular calcium). Thus, a GPCR itself is a GPCR pathway molecule, as is its ligand, as is intracellular calcium.

As used herein “a correlation” refers to a statistically significant relationship determined using routine statistical methods known in the art. For example, in an embodiment, statistical significance is determined using a Student's unpaired t-test, considering differences as statistically significant at p<0.05.

As used herein, a “diagnostic probe” is a probe whose binding to a tissue and/or cell sample provides an indication of the presence or absence of a particular trait. In an embodiment, a probe is considered diagnostic if it binds to a diseased tissue and/or cell (“disease samples”)in at least about 80% of samples tested comprising diseased tissue/cells and binds to less than 10% of non-diseased tissue/cells in samples (“non-disease” samples). Preferably, the probe binds to at least about 90% or at least about 95% of disease samples and binds to less than about 5% or 1% of non-disease samples.

As used herein a “peptide” refers to a biomolecule comprising fewer than 20 consecutive amino acids.

As used herein, a “polypeptide” refers to a biomolecule which comprises more than 20 consecutive amino acids. The term “polypeptide” is meant to encompass proteins, but also encompasses fragments of proteins, or cleaved forms of proteins, or partially digested proteins which are greater than 20 consecutive amino acids.

### Integrated Microfluidic Proteomic Analysis System

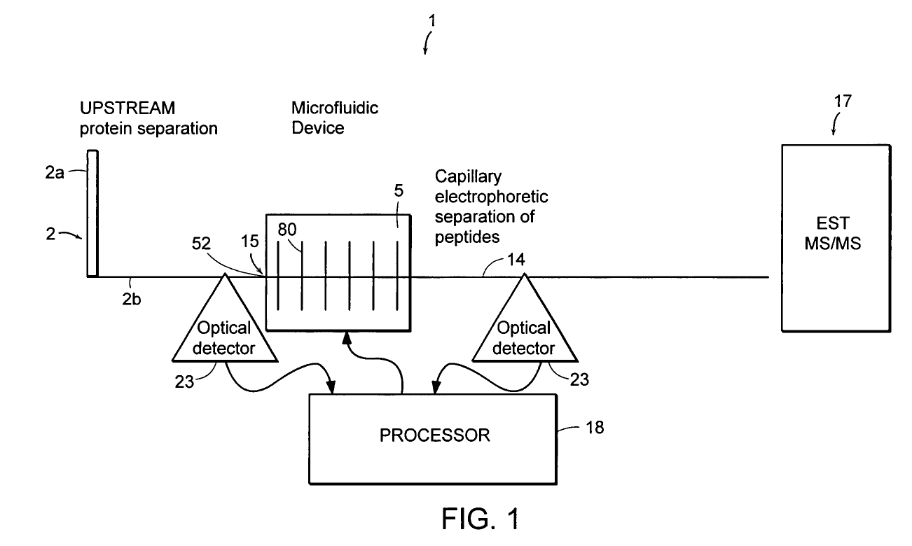

An integrated microfluidic proteomic analysis system of the present invention is shown generally at 1 in FIG. 1. A preferred embodiment of the integrated proteomic analysis system 1 comprises an upstream separation module 2, preferably a multi-dimensional chromatography apparatus including one or more separation columns (e.g., 2a, 2b, etc.) terminating with a capillary electrophoresis separation interfaced with at least one microfluidic device 5. The microfluidic device 5 includes an entrance channel 52 for receiving a substantially purified polypeptide from the upstream separation module 2. In an embodiment of the present invention, the microfluidic device 5 is covered by an overlying substrate (e.g., a coverglass, not shown) which comprises openings communicating with the one or more channels of the microfluidic device 5 and through which solutions and/or reagents can be introduced into the channels. As to be discussed below, Edman degradation takes place on the microfluidic device 5.

As shown in FIG. 4, in an embodiment of the present invention, the microfluidic device 5 comprises a plurality reagent reservoirs 60, 62, 64, 66, and 68. The overlying substrate also maintains the microfluidic device 5 as a substantially contained environment, minimizing evaporation of solutions flowing through the various channels of the microfluidic device 5. In one embodiment, the device comprises open channels.

In a preferred embodiment of the present invention, a substantially purified polypeptide is confined to an at least one reaction channel 80 of a microfluidic device 5. In a preferred embodiment of the present invention, the substantially purified polypeptide undergoes an Edman degradation reaction while confined in the at least one reaction channel 80. The Edman degradation comprises a cleavage reaction producing a cleavage product. In a preferred embodiment of the invention, the cleavage product is a terminal amino acid of the substantially purified polypeptide.

In an embodiment of the present invention, as the cleavage product travels through the reaction channel 80 of the microfluidic device 5, the cleavage product is concentrated in the reaction channel 80 before being removed from the reaction channel 80. In an embodiment of the invention, the microfluidic device 5 is coupled at its downstream end to a downstream separation module 14 (e.g., such as a capillary electrophoresis) which collects the cleavage products and which can further separate the cleavage product from a by-product of the cleavage reaction. In a preferred embodiment, the cleavage product is a single cleaved amino acid produced from a single cycle of an Edman degradation. The cleavage product is sent to the downstream separation module 14 wherein the downstream separation module 14 isolates the single cleaved amino acid. In a preferred embodiment, the downstream separation module is in communication with a processor 18 which identifies the single cleaved amino acid. In a preferred embodiment, a second cycle of the Edman degradation is initiated once the cleaved amino acid of the first cycle has been removed from the reaction channel and has been identified by the processor 18. The cycles of Edman degradation continue until each amino acid of the substantially purified amino acid has been identified or until the signal generated by the cleaved amino acids are below the detection limit.

In an embodiment of the present invention, the downstream separation module 14 is in communication with a peptide analysis module 17 (e.g., an electrospray tandem mass spectrometer or ESI-MS) which is used to collect information relating to the properties of the individual cleavage products.

In an embodiment of the present invention, the integrated microfluidic proteomic analysis system 1 comprises a system processor 18 which can convert electrical signals obtained from different modules of the integrated microfluidic proteomic analysis system 1 (and/or from their own associated processors or microprocessors) into information relating to separation efficacy and the properties of the substantially separated purified polypeptides as they travel through different modules of the system. In an embodiment, the system processor 18 also monitors the rates at which proteins/peptides move through different modules of the system. In an embodiment, signals are obtained from one or more detectors 23 which are in optical communication with different modules and/or channels of the system 1.

The integrated microfluidic proteomic analysis system 1 can vary in the arrangements and numbers of components. For example, the number and arrangement of detectors 23 can vary. In an embodiment, the microfluidic device 5 can interface directly with the peptide analysis module 17 without connection to an intervening downstream separation module 14. In another embodiment, the microfluidic device 5 also can perform separation, eliminating the need for one or more separation functions of the upstream separation module 2. In an embodiment, a digested or partially digested substantially purified polypeptide can be delivered to the microfluidic device 5 after being obtained from a protease digestion device not connected to the integrated proteomic analysis system 1, or in a less preferred embodiment, after being obtained from an on-gel digestion process.

In another embodiment, although the integrated proteomic analysis system 1 is described as being “integrated” in the sense that the different modules complement the other modules' functions, various components of the integrated microfluidic proteomic analysis system 1 can be used separately and/or in conjunction with other systems. In an embodiment, components selected from the group consisting of: the upstream separation module 2, the microfluidic device 5, and downstream separation module 14, and combinations thereof, can be used separately. In another embodiment, some modules can be repeated within the integrated proteomic analysis system 1, e.g., there may be more than one upstream and/or downstream separation module (2 and/or 14), more than one microfluidic device 5, more than one detector 23, and more than one peptide analysis module 17 within the integrated microfluidic proteomic analysis system 1. It should be obvious to those of skill in the art that many permutations are possible and that all of these permutations are encompassed within the scope of the present invention.

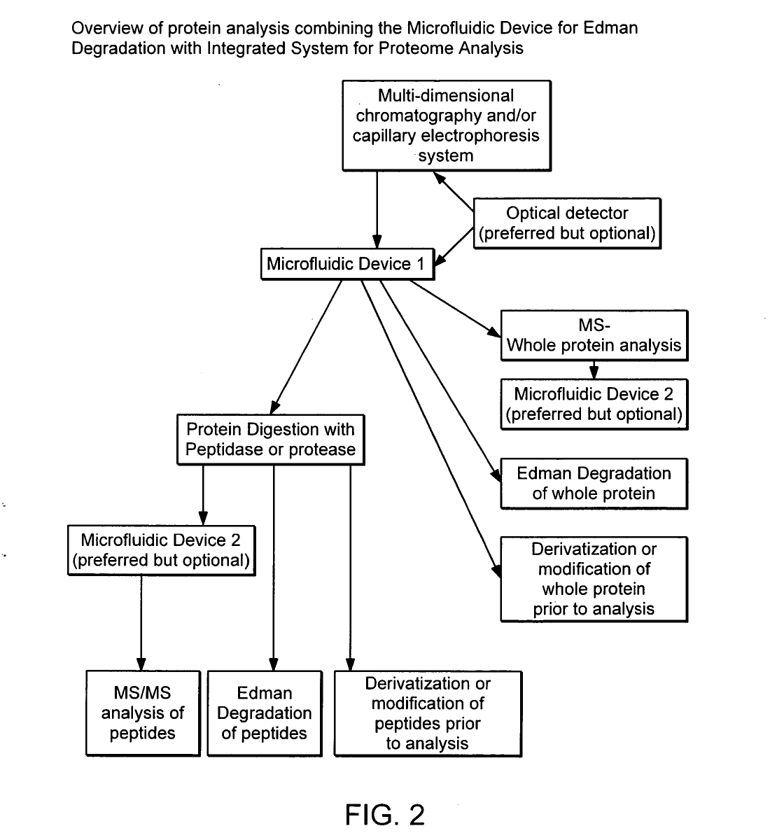

As shown in FIG. 2, the present invention may be used in conjunction with “Microfluidic System For Proteome Analysis”, as disclosed in the Assignee's co-pending Provisional Patent Application, U.S. Serial No. 60/344,456. As shown, in one embodiment, the present invention may be used to perform Edman degradation on a substantially purified polypeptide delivered to the microfluidic device via an upstream separation module 2. In another embodiment, the substantially purified polypeptide is first digested on a first microfluidic device and subsequently delivered to a second microfluidic device 5 capable of performing Edman degradation on the partially digested protein.

### Upstream Separation Module

In a preferred embodiment of the present invention, the upstream separation module 2 comprises a multi-dimensional column separation apparatus. In multi-dimensional separations, samples are separated in at least two-dimensions in accordance with different criteria. For example, in a first dimension, components in a sample may be separated using isoelectric focusing providing information relating to the isoelectric point of a component of interest and in the second dimension, components having the same isoelectric point can be separated further according to molar mass.

As shown in FIG. 1, the upstream separation module 2 of the invention comprises at least a first separation path 2a and a second separation path 2b. In an embodiment, at least one of the separation paths is a capillary. In another embodiment, both separation paths are capillaries. The first separation path 2a and second separation path 2b comprise a first and a second separation medium.

In another embodiment of the invention, the first separation path is a capillary coupled to an injection apparatus (e.g., such as a micropipettor, not shown) which injects or delivers a sample including a mixture of polypeptides to be separated into the first separation medium. In a preferred embodiment of the invention, a sample comprises a lysate of cell(s), tissue(s), organism(s) (e.g., microorganisms such as bacteria or yeast) and the like. In a preferred embodiment of the present invention, a sample comprises a lysate of abnormally proliferating cells (e.g., such as cancerous cells from a tumor). The sample also can comprise subcellular fractions such as those which are enriched for particular organelles (e.g., such as nuclei or mitochondria). In an embodiment of the present invention, the proteins are concentrated prior to separation. In a preferred embodiment, the sample which is injected into the first separation medium comprises micrograms of polypeptides.

One or more electrodes (not shown) coupled at least at a first and second end of the first separation path 2a is used to create an electric field along the separation path. In an embodiment of the invention, a second separation path 2b connects to the first separation path 2a, receiving samples from the first separation path 2a which have been substantially separated according to a first criteria. Passage of the separated samples through the second separation path 2b substantially separates these samples according to a second criteria. Multiple parallel separation paths 2b also can be provided for separating samples in parallel. Systems and methods for controlling the flow of samples in separation paths are described in U.S. Pat. No. 5,942,093.

The region of intersection of the first and the second separating paths, 2a and 2b, respectively, forms an injection apparatus for injecting the sample substantially separated according to the first criteria into the second separation medium. If capillary electrophoresis is used for the separation 2b, an electric field applied along the second separating path 2b then causes the samples substantially separated according to the first criteria to become substantially separated according to the second criteria. In an embodiment of the invention, one or more waste paths (not shown) are provided to draw off unwanted carrier medium (see, e.g., as described in U.S. Pat. No. 5,599,432).

Additional separation paths can be provided downstream of the first separation path 2a, for example, connected to the second separation path 2b or between the first separation path 2a and the second separation path 2b. Each of these additional paths can perform separations using the same or different criteria as upstream separation paths.

In an embodiment of the present invention, at least one separation medium in at least one separation path is used to establish a pH gradient in the path. In an embodiment, ampholytes can be used as the first separation medium. The first separation path 2a can be connected at one end to a reservoir portion (not shown) and at the other end to a collecting path (not shown) proximate to the intersection point between the first and second path. Electrodes can be used to generate an electric field in a reservoir including the ampholyte and in the collecting path. The acidic and basic groups of the molecules of the ampholyte will align themselves accordingly in the electric field, migrate, and in that way generate a temporary or stable pH gradient in the ampholyte.

Different separating paths, reservoirs, collecting paths, and waste paths can be isolated from other paths in the upstream separation module 2 using valves operating in different configurations to either release fluid into a path, remove fluid from a path, or prevent fluid from entering a path (see, e.g., as described in U.S. Pat. No. 5,240,577, the entirety of which is hereby incorporated by reference). Controlling voltage differences in various portions of the upstream separation module 2 also can be used to achieve the same effect. In a preferred embodiment, the opening or closing of valves or changes in potential is controlled by the processor 18, which is further in communication with one or more detectors 23 which monitors the separation of components in different paths within the upstream separation module 2 (see, e.g., as described in U.S. Pat. No. 5,240,577).

In this way, the first separating path 2a can be used to perform isoelectric focusing while the second separating path 2b can be used to separate components by another criteria such as by mass. It should be obvious to those of skill in the art that isoelectric focusing also could be performed in the second path 2b while separation by mass could be performed in the first path by changing the configuration of the reservoir and collecting path. In another embodiment of the present invention, multiple different pH gradients can be established in multiple different separation paths in the upstream separation module 2.

The choice of buffers and reagents in the upstream separation module 2 will be optimized to be compatible with a downstream system with which it connects, such as a microfluidic device 5 which can perform Edman degradation of the substantially purified polypeptides (described further below). In a preferred embodiment, a buffer is selected which maintains polypeptide/peptide solubility while not substantially affecting reactions occurring in the downstream system (e.g., such as cleavage and ultimately, amino acid analysis). In an embodiment, low concentrations of acetonitrile (ACN) and solubizing agents such as urea and guanidine can be used as these will not affect analyses such as ionization (such as would occur in the downstream peptide analysis module 17). When a CE column is used as an upstream separation module 2, a solid-phase extraction (SPE) CE system that incorporates an SPE bead can be provided upstream of the CE column, enabling buffers to be changed and samples to be concentrated prior to CE separation. Commercially available chromatography beads have been designed specifically for the extraction of proteins from detergent containing solutions (Michrom Bioresources, Auburn, Calif.). Elution from the SPE also can achieved with ACN.

In a preferred embodiment of the invention, at least one separation is performed which relies on size-exclusion, e.g., such as size-exclusion chromatography (SEC) (see, e.g., Guillaume, et al., 2001, Anal. Chem. 73(13): 3059-64). Ion-exchange also can be employed and has the advantage of being a gradient technique. Both of these separations are compatible with the surfactants and denaturants used to maintain protein solubility. In another embodiment of the invention, at least one separation is a chromatofocusing (CF) separation. CF separates on the basis of isoelectric point (pI) and can be used to prepare milligram quantities of proteins (see, e.g., Burness et al., 1983, J. Chromatogr. 259(3): 423-32; Gerard et al., 1982, J. Immunol. Methods 55(2): 243-51. In a preferred embodiment, SEC is performed in the first separating path 2a, and CF is performed in the second separating path 2b, achieving a level and quality of separation similar to 2DE.

Parallel separations can be incorporated readily into the integrated microfluidic proteomic analysis system 1 according to the invention, as a microfluidic device 5 including up to about 96 channels or more have been fabricated (see, as described in, Simpson et al., 1998, Proc. Nat. Acad. Sci. USA 95: 2256-2261; Liu et al., 1999, Analytical Chemistry 71: 566-573, for example).

Because the upstream separation module 2 preferably is used to concentrate macrovolumes (i.e., microliters vs. nanoliters) including micrograms of sample, it is preferred that at least one component of the upstream separation module 2 be able to concentrate macrovolume samples and separate polypeptides within such sample. In a preferred embodiment of the invention, therefore, the upstream separation module 2 comprises one or more chromatography columns, preferably, at least one capillary electrochromatography column.

In an embodiment, the separation path can comprise a separation medium including tightly packed beads, gel, or other appropriate particulate material to provide a large surface area over which a fluid including the sample components can flow. The large surface area facilitates fluid interactions with the particulate material, and the tightly packed, random spacing of the particulate material forces the liquid to travel over a much longer effective path than the actual length of the separation path. The components of a sample passing through the separation path interact with the stationary phase (the particles in the separation path) as well as the mobile phase (the liquid eluent flowing through the separation path) based on the partition coefficients for each of the components in the fluid. The partition coefficient is a defined as the ratio of the concentration of a component in a stationary phase to the concentration of a component (e.g., a polypeptide or peptide) in a mobile phase. Therefore, components with large partition coefficients migrate more slowly through the column and elute later.

In a preferred embodiment of the invention, chromatographic separation in the upstream separation module 2 is facilitated by electrophoresis. Preferably, the separation occurs in tubes such as used in capillary electrochromatography (CEC).

CEC combines the electrically driven flow characteristics of electrophoretic separation methods with the use of solid stationary phases typical of liquid chromatography, although smaller particle sizes are generally used. It couples the separation power of reversed-phase liquid chromatography with the high efficiencies of capillary electrophoresis. Higher efficiencies are obtainable for capillary electrochromatography separations over liquid chromatography. In contrast to electrophoresis, capillary electrochromatography is capable of separating neutral molecules due to analyte partitioning between the stationary and mobile phases of the column particles using a liquid chromatography separation mechanism.

In CEC, the stationary phase can be either particles which are packed into capillary tubes (packed CEC) or can be attached (i.e., modified or coated) onto the walls of the capillary (open tubular or OTEC). The stationary phase material is similar to that used in micro-HPLC. The mobile phase, however, is pumped through the capillary column using an applied electric field to create an electro-osmotic flow, similar to that in CZE, rather than using high pressure mechanical pumps. This results in flat flow profiles which provide high separation efficiencies. Therefore, in a currently preferred embodiment of the present invention, at least one component of the upstream separation module 2 comprises one or more CEC columns.

CEC systems can also be provided as part of a microfluidic device. See, as described in Jacobson et al., 1994, Anal. Chem. 66: 2369-2373, for example.

### Microfluidic Device For Edman Degradation of a Peptide Or Polypeptide

Microfluidic devices have been developed for rapid analysis of large numbers of samples. Compared to the prior art, a microfluidic based separation and sample processing device has higher sample throughput, reduced sample and reagent consumption and reduced chemical waste. The liquid flow rates for microfluidic based separation devices range from approximately 1-300 nanoliters (nL) per minute for most applications.

A microfluidic device offers new methods for handling small volume solutions without dilution. The compact format of the microfluidic device apparatus allows for the massive parallelism required for proteome analysis. Arrays of up to 96 capillaries have been fabricated on devices for high throughput DNA sequencing (Simpson et al., 1998, supra; Liu et al., 1999, supra). Further, on-device electroosmotic pumping of a sample through different channels of a microfluidic device can be achieved simply with arrays of electrodes. Controlling an electrode array is much simpler than controlling an array of high pressure lines and valves. Additionally, the closed system architecture reduces contamination and difficulties caused by evaporation.

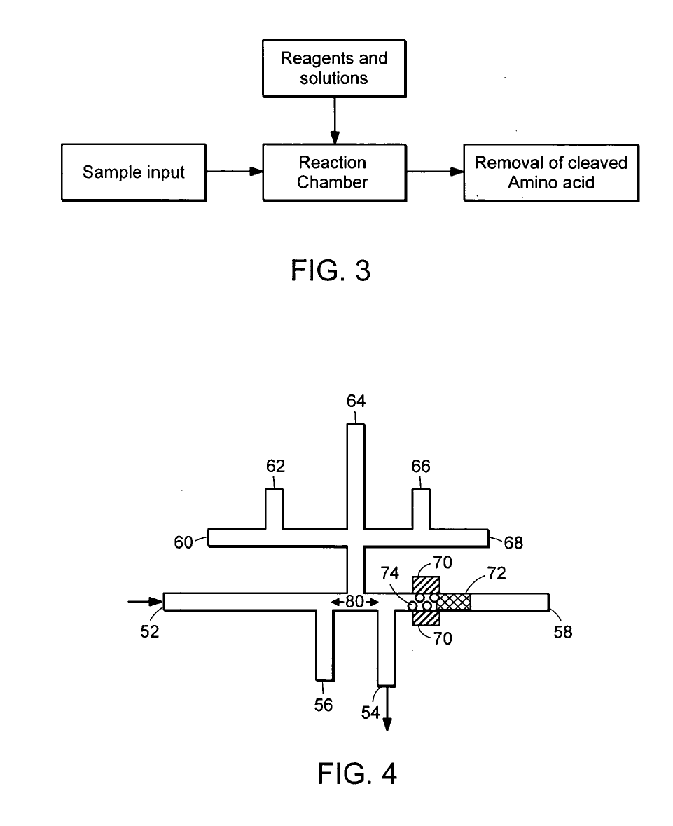

As shown in FIG. 1, an embodiment of the integrated proteomic analysis system 1 of the present invention comprises a microfluidic device downstream of the upstream separation module 2. The microfluidic device 5 is in communication with the upstream separation module 2 through an entrance channel interface 15 which comprises an entrance channel 52 for connecting to one or more separating paths of the upstream separation module 2. The upstream separation module 2 delivers a substantially purified polypeptide to the microfluidic device 5 via the entrance channel 52. FIG. 3 shows a box diagram of the general relationships of the components of the microfluidic device 5. The microfluidic device 5 of the present invention is capable of performing Edman degradation.

In a preferred embodiment of the present invention, the microfluidic device 5 comprises a biocompatible substrate such as silicon or glass and the microfluidic device 5 comprises a plurality of reaction channels 80. Preferably, the microfluidic device 5 comprises at least about 2, at least about 4, at least about 8, at least about 16, at least about 32, at least about 48, or at least about 96 reaction channels 80. Reaction channels 80 can vary in size and are generally from about 50 μm-200 μm wide (preferably, from about 80 μm-100 μm wide) and from about 5 μm-40 μm deep (preferably from about 10 μm-30 μm deep). The microfluidic device 5 is not necessarily planar and may be represented in a three-dimensional channel network. In a most preferred embodiment, the microfluidic device 5 is circular in shape.

The microfluidic device 5 may comprise varying channel geometries. In an embodiment, the microfluidic device 5 comprises an entrance channel 52 which divides into a plurality of substantially parallel reaction channels 80. However, the absolute channel geometry is not critical so long as the appropriate fluid flow relationships are maintained. In an embodiment, the various channels may be curved. In an embodiment, the substrate itself is not planar and the various channels may be non-coplanar (e.g., radiating from a central intersection channel as spokes from a central hub). Many refinements to the geometry of the channel layout can be made to increase the performance of the device and such refinements are encompassed within the scope of the invention. In an embodiment, shorter channels will decrease the distance over which sample bands must be transported, but generally channels need to be long enough to hold the sample bands, and to provide adequate separation between electrodes in contact with channels (discussed further below) to prevent current feedback.

The microfluidic device 5 can be substantially covered with an overlying substrate for maintaining a substantially closed system (e.g., resistant to evaporation and sample contamination) (not shown). The overlying substrate can be substantially the same size as the microfluidic device 5, but at least is substantially large enough to cover the reaction channels 80 of the microfluidic device 5. In an embodiment of the invention, the overlying substrate comprises at least one opening for communicating with the microfluidic device 5. The openings can be used to add reagents or fluids to the microfluidic device 5. In a preferred embodiment of the invention, openings can be used to apply an electric voltage to different channels in communication with the openings.

Suitable materials to form the overlying substrate comprise silicon, glass, plastic or another polymer. In an embodiment of the invention, the overlying substrate comprises a material which is substantially transmissive of light. The overlying substrate can be bonded or fixed to the microfluidic device 5, such as through anodic bonding, sodium silicate bonding, fusion bonding as is known in the art or by glass bonding when both the microfluidic device 5 and overlying substrate comprise glass (see, e.g., as described in High Technology, Chiem et al., 2000, Sensors and Actuators B 63: 147-152).

As shown in FIG. 4, an embodiment of the invention comprises a microfluidic device 5 accepting a substantially purified polypeptide from an upstream separation module via an entrance channel 52. In a preferred embodiment of the invention, a reaction channel 80 engages the entrance channel 52 wherein the substantially purified polypeptide is delivered to the reaction channel 80. Once the substantially purified polypeptide enters the reaction channel 80, the substantially purified polypeptide is confined to the reaction channel 80.

In an embodiment of the present invention, a solid support inside the reaction channel 80 engages the substantially purified polypeptide thereby confining the substantially purified polypeptide to the reaction channel 80. The substantially purified polypeptide is confined to the reaction channel 80 through immobilization on a solid support which is also confined to the reaction channel 80. Because the solid support is orders of magnitudes larger in size than the substantially purified polypeptide, attachment of the substantially purified polypeptide to the solid support facilitates confining the substantially purified polypeptide to the reaction channel 80.

In an embodiment of the present invention, the solid support is a membrane 72. In an embodiment of the present invention, the solid support is a poly-vinylidene flouride (“PVDF”) membrane 72. In another embodiment of the present invention, the membrane is a cellulose membrane 72.

In a preferred embodiment of the invention, the solid support is an ultrafiltration membrane 72. Ultrafiltration is a membrane process which will retain soluble macromolecules and every thing larger while passing solvent, ions, and other small soluble species. Ultrafiltration is almost always operated with some means of forced convection near the membrane. Cross-flow filtration is practically universal for ultrafiltration.

In a preferred embodiment of the present invention, an ultrafiltration membrane 72 confines a substantially purified polypeptide to the reaction channel 80. In another embodiment, the substantially purified polypeptide is not immobilized on a solid support. The substantially purified polypeptide remains in solution in the reaction channel 80. In an embodiment, a cleavage product is allowed to pass through the ultrafiltration membrane 72 while the ultrafiltration membrane 72 confines the substantially purified polypeptide to the reaction channel 80.

In a preferred embodiment of the invention, the solid support comprises a plurality of beads 74. The plurality of beads 74 can be divided into two types: magnetic and non-magnetic. A supplier of the magnetic beads is Dynal Biotech. The non-magnetic beads are available from numerous sources who supply beads for chromatography.

The plurality of beads 74 can be packed into a reaction channel 80 of a microfluidic device 5 by applying voltages at selected channels to drive the plurality of beads 74 into the desired reaction channels 80. In an embodiment, the plurality of beads 74 comprise charged surface molecules (e.g., such as free silonol groups) to facilitate the process of packing the plurality of beads 74 into a reaction channel 80. For example, electroosmotic flow driven by walls of the reaction channel 80 and free silonol groups on the plurality of beads 74 can be used to effect packing. In an embodiment, a voltage of from about 200-800 V for about 5 minutes at a reaction channel 80 while remaining, non-selected channels are grounded, is sufficient to drive the plurality of beads 74 into a selected reaction channel 80. Packing of a plurality of beads 74 also may be performed electrokinetically as described in U.S. Pat. No. 5,942,093, which is hereby incorporated by reference.

In an embodiment of the invention, using bead injection technology for the addition and removal of the plurality of beads 74 from the reaction channel 80 allows for the introduction of new beads that are activated for a covalent attachment of the substantially purified polypeptide and will result in minimal carry-over (see, e.g., Ruzicka and Scampavia, 1999 Anal. Chem. 71(7): 257A-263A; Oleschuk et al., 2000, Anal. Chem. 72(3): 585-590).

In another embodiment of the invention, the plurality of beads 74 are magnetic, paramagnetic or superparamagnetic, and can be added to or removed from a reaction channel 80 of the microfluidic device 5 by using a magnetic field applied to selective regions of the microfluidic device 5.

In a preferred embodiment of the invention, the substantially purified polypeptide engages to the plurality of beads 74. For standard silica chromatography beads the same chemistry can be used to engage the plurality of beads 74 to the substantially purified polypeptide as was disclosed in Aebersold et al (Analytical Biochemistry, 56-65, 1990). Aebersold discloses using N-ethyl-N′-(3-dimethylaminopropyl) carbodiimide (EDC) to activate the carboxylic acid terminus and aminophenyltriethoxysilane (APTE) to activate the silica surface. Other activated beads which can bind covalently to a carboxylic acid group and can be used for N-terminal sequencing, and any activated bead which can bind covalently to a primary amine can be used for C-terminal sequencing. Both covalent and non-covalent methods for immobilization of the substantially purified polypeptide are known in the art.

In an embodiment of the invention, the substantially purified polypeptide is engaged to the plurality of beads 74 at a C-Terminal end of the plurality of polypeptides. In another embodiment of the invention, the substantially purified polypeptide is engaged to the plurality of beads 74 at a N-Terminal end of the substantially purified polypeptide. In a preferred embodiment of the invention, the substantially purified polypeptide is covalently bonded to the plurality of beads 74.

In a preferred embodiment of the present invention, the plurality of beads 74 are confined to the reaction channel 80. In an embodiment of the invention, the plurality of beads 74 are magnetic. In an embodiment, an external force is applied to the plurality of beads 74 in order to confine the plurality of magnetic beads 74 to the reaction channel 80. In another embodiment, a plurality of magnets 70 are used to confine the plurality of magnetic beads 74 to the reaction channel 80.

In another embodiment of the present invention, the reaction channel 80 comprises a blocking structure which blocks the solid support from exiting the reaction channel 80. The blocking structures can be classified into two main groups that are differentiated based on the size regimes of the pores or openings in the blocking structure.

In an embodiment of the present invention, a weir confines the plurality of beads 74 to the reaction channel 80. Weirs, posts, constricters, and filters fabricated in the reaction channel 80 are in the larger class of structures that will block the plurality of beads 74, but do not impede the flow of a cleavage product or the substantially purified polypeptide. Liquid flow in an open channel may be slowed by means of a weir, which consist of a dam over which, or through a notch in which, the liquid flows. The terms “rectangular weir,” “triangular weir,” etc., generally refer to the shape of the notch in a notched weir.

In a preferred embodiment of the invention, an ultrafiltration membrane 72 is used to confine the plurality of beads 74 to the reaction channel 80. An ultrafiltration membrane is a member of the second class of smaller structures. The second class of smaller structures impedes the movement of the plurality of beads 74, the substantially purified polypeptide and sometimes the cleavage product. Ultrafiltration membranes are commercially available in different materials. In an embodiment of the invention, an ultrafiltration membrane 72 is incorporated into the reaction channel 80 for the purpose of confining a plurality of beads 74 to the reaction channel 80. In another embodiment, an ultrafiltration membrane 72 confines a plurality of beads 74 and the substantially purified polypeptide as well as a cleavage product to the reaction channel 80.

In a preferred embodiment of the present invention, a plurality of magnets 70 and an at least one ultrafiltration membrane 72 are used together in order to confine a plurality of beads 74 to the reaction channel 80.

The microfluidic device 5 of the present invention is capable of various configurations. FIG. 4 shows an embodiment of the present invention wherein a substantially purified polypeptide enters the reaction channel 80 via an entrance channel 52 which is in communication with an upstream separation module 2. Once inside the reaction channel 80, the substantially purified polypeptide is engaged to a plurality of magnetic beads 74. The plurality of magnetic beads 74 are confined to the reaction channel 80 by applying an external force. In the embodiment of FIG. 4, a plurality of magnets 70 apply the external force to the plurality of magnetic beads 74.

As shown in FIG. 4, the present invention can further comprise an ultrafiltration membrane 72. The plurality of magnetic beads 74, the substantially purified polypeptide, and the cleavage products are all concentrated at the ultrafiltration membrane 72. The ultrafiltration membrane 72 impedes the flow of the plurality of magnetic beads 74, the substantially purified polypeptide, and the cleavage products. Once the cleavage product has been concentrated at the ultrafiltration membrane 72, the cleavage product leaves the reaction channel 80 via the exit channel 54. FIG. 4 also illustrates a channel 56 for the introduction and for the removal of a plurality of beads 74 and a connection 58 to an auxiliary electrode.

FIG. 4 also shows a plurality of channels which connect to the reagent reservoirs 60, 62, 64, 66, and 68 wherein each reagent reservoir engages the reaction channel 80. In an embodiment, each reagent reservoir 60, 62, 64, 66, and 68 contains a unique reagent. In a preferred embodiment of the present invention, each unique reagent is critical to a step of Edman degradation. When covalent coupling of the polypeptide is performed, a coupling reagent, such as EDC, would occupy one of the reservoirs.

In an embodiment of the present invention, the plurality of reagents are driven to the reaction channel 80 by applying a centrifugal force to the microfluidic device 5. The reagents will be driven from a reagent reservoir 60, 62, 64, 66, 68 to the reaction channel 80 at a rate proportional to the centrifugal force (see, e.g., Duffy et al., 1999, Analytical Chemistry 71, 4669-4678). The flow of reagents from the reagent reservoirs 60, 62, 64, 66, 68 will control the rate and progress of the reaction.

In one embodiment of the present invention, the reagents are mixed while being driven from the reagent reservoir 60, 62, 64, 66, 68 to the reaction channel 80. Mixing of reagents may lead to a quicker and more efficient cleavage reaction. Mixing occurs by allowing more than one reagent from more than one reagent reservoir 60, 62, 64, 66, 68 to come into fluid contact with one another prior to reaching the reaction channel 80. In one embodiment, a passage channel engages the reaction channel 80 and a plurality of reaction reservoirs 60, 62, 64, 66, 68. The passage channel allows a reagent to travel from the reagent reservoir 60, 62, 64, 66, 68 to the reaction channel. In one embodiment, centrifugal force drives a first reagent from a first reagent reservoir 60, 62, 64, 66, 68 into a passage channel which leads to the reaction channel 80. A second reagent is driven from a second reagent reservoir 60, 62, 64, 66, 68 and enters the passage channel leading to the reaction channel 80. Once the second reagent enters the passage channel, the second reagent comes into fluid communication with the first reagent. The amount of mixing is controlled by the length of the passage channel leading to the reaction channel 80 and the amount of centrifugal force (see, e.g., Duffy et al., 1999, Analytical Chemistry 71, 4669-4678). In a preferred embodiment, the microfluidic device 5 will contain a plurality of passage channels.

In one embodiment of the present invention, mixing of reagents can occur within the reaction channel 80. In this embodiment, a plurality of magnetic beads 74 are placed in the reaction channel 80 and are activated by applying an outside force to the plurality of beads 74. A magnet 70 can be used to apply the outside force. Once activated, the plurality of magnetic beads 74 may vibrate and/or move inside the reaction channel 80 and provide for mixing of reagents.

In a preferred embodiment of the present invention, an electric field is applied to induce electroosmotic and electrophoretic flow.

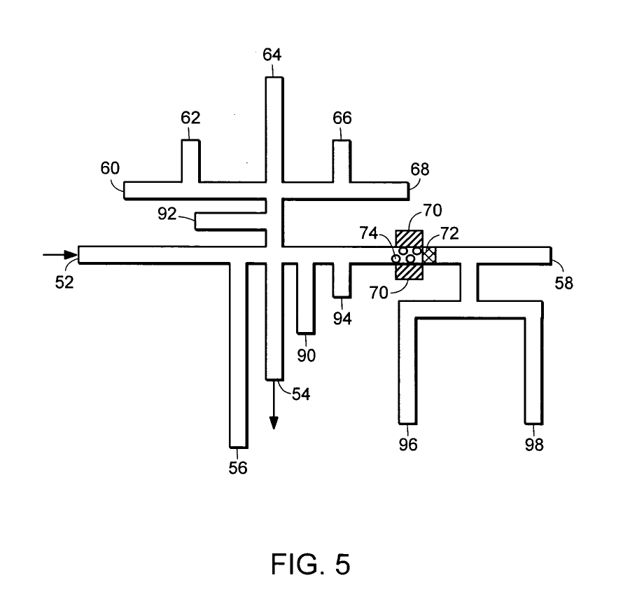

As shown in FIG. 5, an embodiment of the present invention comprises the aspects discussed with FIG. 4 and further includes channels to facilitate an electroosmotic flow (“EOF”) pump. FIG. 5 further shows an embodiment of the invention further comprising a first waste stream 90 and a second waste stream 92. In one embodiment, a trifluoroacetic acid (“TFA”) channel 94 is provided for the addition of TFA to the reaction channel 80. Flow from the first waste stream 90 to the second waste stream 92 during EOF pumping of TFA from the TFA channel 94 through the membrane 72 prevents contamination of TFA from upstream reagents and solutions. In one embodiment, an EOF Pump regulates fluid flow. A first reservoir 96 and a second reservoir 98 are connected to the channels which generate the fluid flow by EOF.

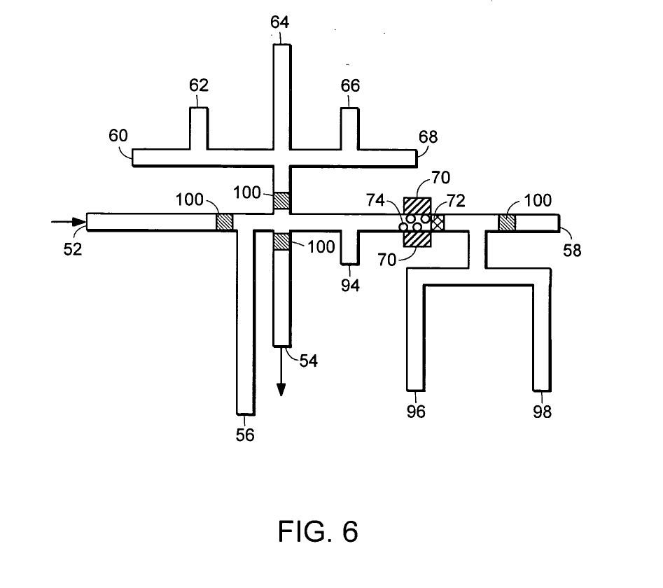

Since these channels are pulling solution from inside the reaction channels, the flow inside the reaction channels will be hydrodynamic flow which will pull solution from all of the intersecting channels. As shown in FIG. 6, a plurality of flow restrictors may be used to minimize the contribution of flow from these intersecting channels. Minimizing flow from the intersecting channels will improve the purity of the TFA in the reaction channel. The flow restrictors can be many small channels in parallel or a macroporous frit-like material. Such macroporous materials will allow for the reagents and large molecules to move through them while increasing the resistance to hydrodynamic flow.

As opposed to the prior art, the EOF pump region (where the electric field is) of the present invention is not co-linear with field free or hydrodynamic pumping region as is usually the case. The positive and negatively charged surface regions generate flow in opposite directions and can be used to pump solution into or out of the field free channel, tube, capillary, or hose; a number of channels, tubes, capillaries, or hose in parallel, or a packed bed or porous material held inside a column where the material has a charged surface. In a preferred embodiment, the positive and negative charger regions are filled with a porous material. Filling the channels with a porous material increases the ability of the pump to pump against the hydrodynamic back pressure that it will generate. This resistance to backpressure of the small channels is due to the fact that the volumetric flow rate for hydrodynamic (pressure driven) flow through an open tube is inversely proportional to (the radius)\hat{ }4. The packed bed approximates an array of parallel channels with very small internal diameters. Changing the direction of the hydrodynamic flow in the field free region is achieved by simply changing the polarity of the voltage on the high voltage power supply. It is often detrimental to have the electrode in the channel because it creates bubbles (hydrogen or oxygen) from electrolysis of water, and it can produce other unwanted chemical reactions, such as modification of reactants or sample.

This pump can be used to pump solutions that do not pump well using EOF. These solutions would include non-polar organic solvents that do not generate much EOF, and strong acids or bases or salts where the anions and cations would be pumped at different rates. This EOF pump will be applied to the Edman degradation microfluidic device 5 to pump neat trifluoroacetic acid into and out of the reaction channel. If the reaction channel was not electric field free, then the hydrogen ions and the counterions would have different rates of migration, and free hydrogen and hydronium ions would reach the reaction chamber first having a detrimental affect on the selective cleavage. The reaction would take place in the reaction field free channel which is when the EOF pump is used.

FIG. 6 shows an embodiment of the present invention as depicted in FIG. 5 further comprising a plurality of hydrodynamic flow restrictors 100. A hydrodynamic flow restrictor 100 prevents contamination of TFA from upstream reagents and solutions during EOF pumping from the TFA channel 94 through the polypeptide concentrating membrane. The flow restrictors minimize the contribution of flow from these intersecting channels. Minimizing flow from the intersecting channels will improve the purity of the TFA in the reaction channel. The flow restrictors 100 can be many small channels in parallel or a macroporous frit-like material. Such macroporous materials will allow for the reagents and large molecules to move through them while increasing the resistance to hydrodynamic flow.

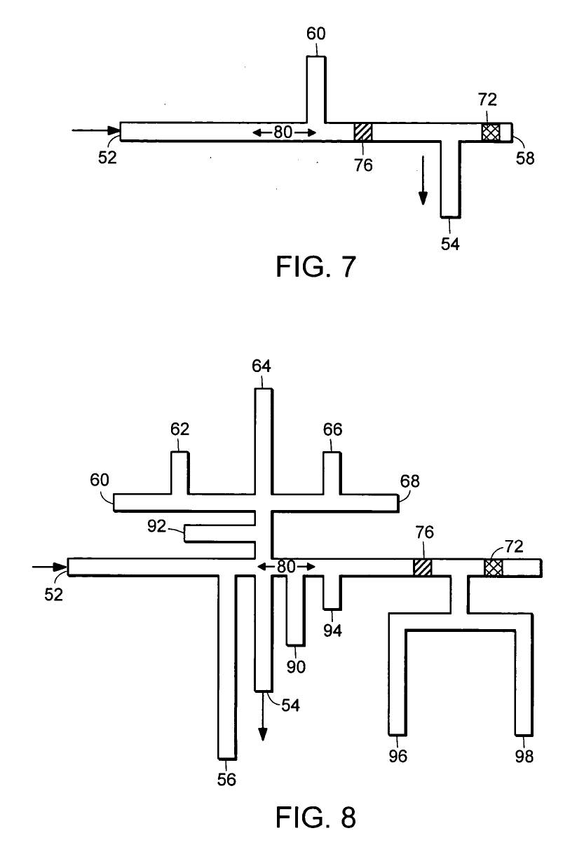

A preferred embodiment of the present invention is shown in FIG. 7. In FIG. 7, the substantially purified polypeptide is confined to the reaction channel 80 by a first ultrafiltration membrane 76. In an embodiment, the substantially purified polypeptide is concentrated at the first ultrafiltration membrane 76. After cleavage of the terminal amino acid of the substantially purified polypeptide, the cleavage products pass through the first ultrafiltration membrane 76 and are concentrated at a second ultrafiltration membrane 72. After the cleavage products are concentrated at the second ultrafiltration membrane 72, the cleavage product is removed from the reaction channel 80 through the exit channel 54. This configuration is similar to 2D but the orientation of the entrance channel 52 and outlet channel 54 have been switched with respect to the membrane 76. This configuration is desirable as the polypeptides will always be pushed towards the membrane 76 to be concentrated. Prior to elution to the detector, the cleaved amino acids are concentrated at a second membrane 72.

In FIG. 8, the substantially purified polypeptide is confined to the reaction channel 80 by a first ultrafiltration membrane 76. In an embodiment, the substantially purified polypeptide is concentrated at the first ultrafiltration membrane 76. After cleavage of the terminal amino acid of the substantially purified polypeptide, the cleavage product passes through the first ultrafiltration membrane 76 and are concentrated at a second ultrafiltration membrane 72. After the cleavage products are concentrated at the second ultrafiltration membrane 72, the cleavage product is removed from the reaction channel 80 through the exit channel 54. FIG. 8 further shows an embodiment of the invention further comprising an EOF Pump (described above in relation to FIG. 6).

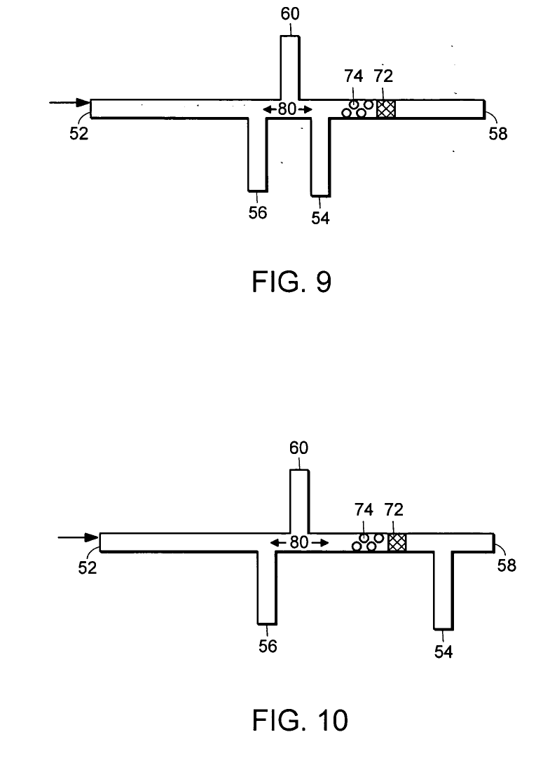

As shown in FIG. 9, the present invention comprises a substantially purified polypeptide engaged to a plurality of beads 74 in a reaction channel 80 of the microfluidic device 5. The plurality of beads 74 are confined to the reaction channel 80 by a blocking structure. In an embodiment, the blocking structure is an ultrafiltration membrane 72. The ultrafiltration membrane 72 impedes the flow of the plurality of beads 74, the flow of the substantially purified polypeptide, and the flow of the cleavage product. In an embodiment, the cleavage product is concentrated at the ultrafiltration membrane 72 before the cleavage product is removed from the reaction channel 80 through the exit channel 54.

As shown in FIG. 10, the present invention comprises a blocking structure wherein the blocking structure does not impede the flow of cleavage products. In FIG. 10, the blocking structure is an ultrafiltration membrane 72. The ultrafiltration membrane 72 confines a plurality of beads 74 to the reaction channel 80 of a microfluidic device 5. The ultrafiltration membrane 72 impedes the flow of the plurality of beads 74 and the flow of the substantially purified polypeptide. The ultrafiltration membrane 72 of an embodiment does not impede the flow of the cleavage product. In an embodiment, the cleavage product passes through the ultrafiltration membrane 72 before the cleavage product is removed from the reaction channel 80 through the exit channel 54. In an embodiment, the ultrafiltration membrane 72 serves to concentrate the cleavage product before allowing the cleavage product to pass through the ultrafiltration membrane 72 and leave the reaction channel 80 via the exit channel 54.

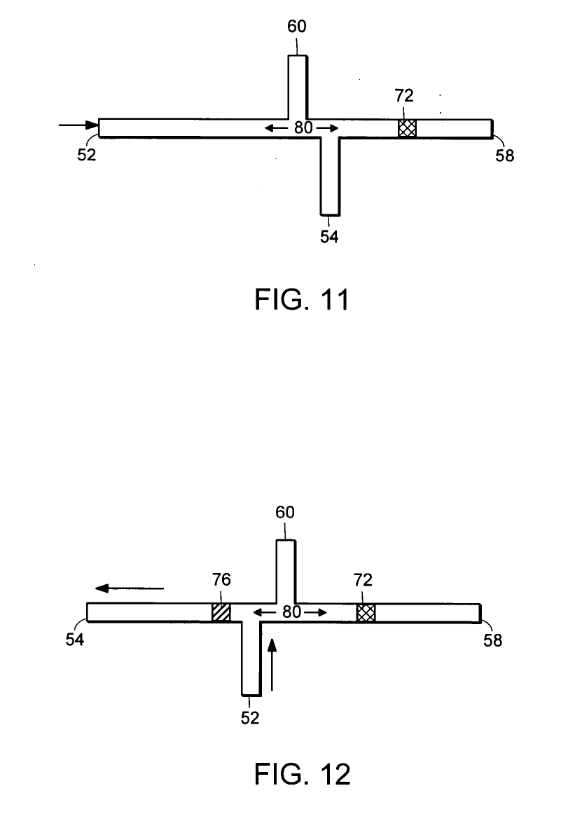

As shown in FIG. 11, the substantially purified polypeptide is confined to the reaction channel 80 by an ultrafiltration membrane 72. In an embodiment, the ultrafiltration membrane 72 impedes the flow of the substantially purified polypeptide and the flow of the cleavage product. In an embodiment, the cleavage product is concentrated at the ultrafiltration membrane 72 before the cleavage product is removed from the reaction channel 80 through the exit channel 54. In this embodiment, polypeptides and cleaved amino acids are concentrated by the ultrafiltration membrane 72. During the reversal of transport to elute the cleaved amino acid the polypeptides will also move away from the membrane 72. Although this movement of the polypeptides is not desirable, it can be tolerated if the transport of the amino acids to the detector is sufficiently fast with respect to movement of the polypeptides, so the cleaved amino acid is eluted through the exit channel 54, but the polypeptide is not. After elution of a cleaved amino acid, a polypeptide would be reconcentrated in front of the membrane 72, before proceeding with the cleavage of the next amino acid.

As shown in FIG. 12, the substantially purified polypeptide is confined to the reaction channel 80 by a first ultrafiltration membrane 72. In an embodiment, the substantially purified polypeptide is concentrated at the first ultrafiltration membrane 72. After cleavage, the cleavage product is concentrated at the ultrafiltration membrane 72. The second ultrafiltration membrane 76 does not impede the flow of the cleavage products, but does impede the flow of the substantially purified polypeptides, ensuring that the purified polypeptide is retained in the reaction channel as the cleavage product is being removed from the reaction channel 80. After the cleavage products pass through the second ultrafiltration membrane 76, the cleavage product is removed from the reaction channel 80 through the exit channel 54.

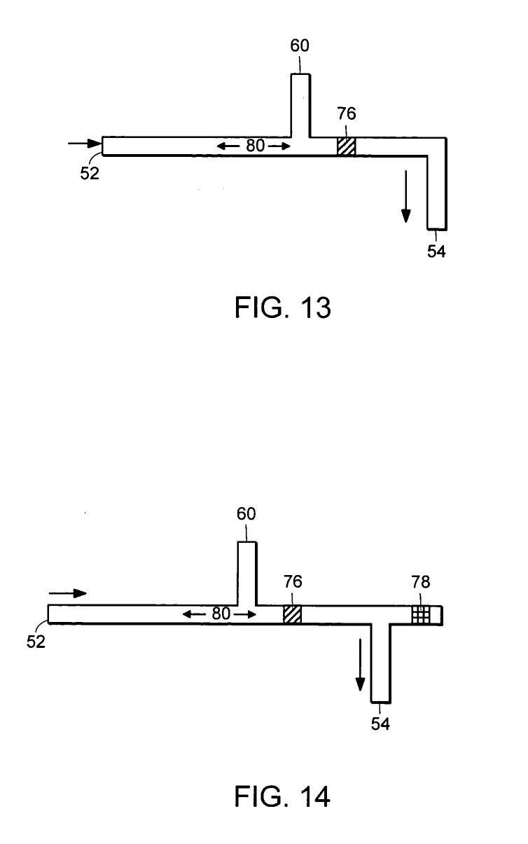

As shown in FIG. 13, the substantially purified polypeptide is confined to the reaction channel by an ultrafiltration membrane 76. In an embodiment, the substantially purified polypeptide is concentrated at the first ultrafiltration membrane 76. After cleavage, the cleavage product passes through the first ultrafiltration membrane 76 and exits the reaction channel 80 via the exit channel 54. The advantage of this embodiment is its ease of use and simplicity, but the disadvantage is that cleavage products of this embodiment are not concentrated before the cleavage products are removed from the reaction channel 80.

As shown in FIG. 14, the substantially purified polypeptide is confined to the reaction channel 80 by an ultrafiltration membrane 76. In an embodiment, the substantially purified polypeptide is concentrated at the first ultrafiltration membrane 76. After cleavage, the cleavage product passes through the first ultrafiltration membrane 76 and are concentrated on a solid phase extraction module 78. Once the cleavage products have been concentrated at the solid phase extraction module 78, the cleavage products exit the reaction channel 80 via the exit channel 54. The solid phase extraction module 78, can be either upstream or downstream of the exit channel 54.

In addition to a plurality of reaction channels 80 for cleavage, additional channels may be provided for the present invention. In an embodiment, one or more channels are provided which are cleavage resistant for moving a substantially purified polypeptide directly to a peptide analysis module 17 to obtain a determination of its mass (e.g., for comparison with cleavage products of the substantially purified polypeptide).

In a preferred embodiment, the microfluidic device 5 comprises at least one electrode in communication with one or more of the various channels in the microfluidic device 5 to drive mass transport of polypeptides through the various channels of the microfluidic device 5. In an embodiment of the invention, flow of solution, including polypeptides, is controlled electroosmotically and electrophoretically by control of voltage through the electrode(s). In an embodiment of the invention, providing a silicon oxide layer on a surface of the microfluidic device 5 provides a surface on which conductive electrodes can be formed (e.g., by chemical vapor deposition, photolithography, and the like). The thickness of the layer can be controlled through oxidation temperature and time and the final thickness can be selected to provide the desired degree of electrical isolation. In a preferred embodiment of the invention, a layer of silicon oxide is provided which is thick enough to isolate electrode(s) from the overlying substrate thereby allowing for the selective application of electric potential differences between spatially separated locations in the different channels of the microfluidic device 5, resulting in control of the fluid flow through the different channels. In aspects where the overlying substrate is not glass, one or more electrodes also can be formed on the overlying substrate.

In a preferred embodiment of the invention, the ends of the channels open into reservoirs. In another embodiment of the invention, one or more electrodes can be in electrical communication with a buffer solution provided in a reservoir well at the terminal end of a reaction channel 80.

In another embodiment of the invention, flow through one or more selected channels of the microfluidic device 5 is hydrodynamic and mediated mechanically through valves placed at appropriate channel junctions as is known in the art. See, e.g., as described in U.S. Pat. No. 6,136,212; U.S. Pat. No. 6,008,893, and Smits, Sensors and Actuators A21-A23: 203 (1990). To improve sample handling and ultimately improve detection limits of the system precise control of flow is required. In an embodiment of the invention, flow of reagents in each of the various channels of the microfluidic device 5 is independently controlled. In an embodiment, transport is voltage driven rather than pressure driven. To prevent or reduce feedback or cross talk between channels, electrodes and buffer reservoirs along undesired alternative paths can be used to block feedback by acting as current and electroosmotic flow drains.

To prevent feedback through connected channels, a series of electrodes can be used that act as either a source or drain of electroosmotic flow. If high currents are passed through the drains, problems can arise from Joule heating or rapid consumption of buffer. Buffer consumption is a technical problem that can be solved by appropriate engineering. Buffer out-gassing, which can occur at high levels of Joule heating can be avoided by degassing buffers before use. The maximum voltage used is largely governed by out-gassing of the buffer solutions used in the system. Since current is proportional to voltage, at higher voltages there will be more Joule heating and a greater tendency for out-gassing to occur. With the current scheme of voltage control for sample transport the largest current will flow between the electrodes that are acting as potential and electroosmotic flow sinks, and these are the areas where outgassing will be most likely. However, very high electric field strengths can be used with microfluidic devices 5 as ultrafast separations have been carried out at 53 kV/cm (see, e.g., Figeys et al., 1997, J. Chromatogr., 763: 295-306) and the present invention contemplates the use of high voltage for rapid sample transport, but an electric field strength below 53 kV/cm.

The voltage that each electrode (represented by the black dots) is held at during each stage of the process is shown by the numbers (absolute values are not important but relative values are). In an embodiment, reservoirs are above the microfluidic device 5 and a small hole is drilled in the overlying substrate to connect the channels and the reservoirs. The distances between adjacent electrodes are equivalent so the voltage at each junction can be easily approximated. When the microfluidic device 5 is made from uncoated, fused silica, the direction of electroosmotic flow will always be from high to low voltage with no voltage drop across parallel channels when parallel channels are present.

As shown in FIG. 1, the microfluidic device 5 collects sample bands including substantially purified polypeptides as they elute from an upstream separation module 2. Preferably, a UV detector 23 located near the recipient channel interface 15 will detect the separated sample bands. The rate at which bands reach this UV detector 23 will be used to compute the mobility of the bands and the time at which the electrode voltage should be modulated on the microfluidic apparatus to direct the flow of sample. When the upstream separation module 2 comprises a capillary electrophoresis apparatus, the electrode switching times can be accurately calculated because the phenomena that give rise to transport are the same phenomena that give rise to transport in the microfluidic device.

In an embodiment, fluid can be directed into one or more reservoirs above the microfluidic device 5 if necessary, so only polypeptide bands are sent to the reaction channels 80. In a preferred embodiment, any running buffer from the upstream separation module 2 between sample peaks that does not contain any sample will be eliminated so it does not take up any space within the microfluidic device 5. Elimination of buffer decreases the amount of time the detector 17 will spend analyzing a sample without peptides, thereby increasing the efficiency of the system 1.

In an embodiment, modulation of the potential at the appropriate electrodes in the array will direct the sample band to the proper channel.

The production of bubbles at electrodes can be problematic. In an embodiment, bubbles will be physically separated from the channels when electrodes are held in the buffer reservoirs above the microfluidic device 5 and where the solution in the reservoirs is connected directly with a channel through a hole in the overlying substrate. If the electrodes are integrated directly onto the channels, then buffer additives can be used to suppress bubble formation, as previously reported for an electrospray MS interface (see, e.g., as described in Moini et al., 1999, Analytical Chemistry 71: 1658-1661).

In an embodiment, where sample channels are in the substantially parallel configuration, electroosmotic pressure induced in the reaction channels 80 through intersection with adjacent reaction channels 80 may slowly force sample bands out and decrease the efficiency of the cleavage process. In an embodiment, by providing an on-device imaging detector 23 (discussed further below) in optical communication with one or more of the reaction channels 80, a user can determine whether sample bands including polypeptides and/or their cleavage products are actually stationary. If they are not stationary, many different methods can be used to counter the effects of this pressure. In an embodiment, electroosmotic flow can be actively controlled by controlling the double layer potential as described by Lee et al., 1990, Anal. Chem. 62: 1550-1552; Wu et al., 1992, Anal. Chem. 64: 886-891; Hayes et al., 1993, Anal. Chem. 65: 27-31; Hayes et al., 1993, Anal. Chem. 65: 2010-2013; and Hayes et al., 1992, Anal. Chem. 64: 512-516. Fabrication of a microfabricated apparatus with such control was recently demonstrated by Schasfoort et al., 1999, Science 286: 942-945.

In an embodiment, electroosmotic pressure in channels having a substantially parallel channel configuration also can be stopped by temporarily breaking electrical contact in the channel. Here, bubbles are desirable and are introduced by low pressure into reaction channel(s) 80 to manipulate flow on the microfluidic device 5. In an embodiment, bubbles can be introduced by physically separating sample plugs or by breaking the electrical conductivity in the channel(s). Strategic positioning of a membrane (e.g., such as a hydrophobic membrane made from polypropylene, polyethylene, polyurethane, polymethylpentene, polytetrafluoroethylene, and the like) which is permeable to the bubbles but not the liquid also can be used for bubble removal. In an embodiment, by allowing gas to pass through, but not solution, such a membrane can be used to direct solution flow. Gas permeable membranes are known in the art and are described in U.S. Pat. No. 6,267,926, for example. In a similar manner, a hydrophobic coating strategically located after a channel intersection can be used for fabrication of on-device passive valves. See, e.g., as described in McNeely et al., 1999, SPIE: Bellingham 3877: 210-220.

The microfluidic device 5 can be optimized to provide the minimum number of electrode controls per microfluidic device 5. In an embodiment, this is accomplished by tying some of the electrodes together. In an embodiment, incorporation of voltage dividers into the circuitry which is part of the microfluidic device 5 can be used to always hold a pair of electrodes at the same relative potential, while their absolute potentials are varied. Such schemes would reduce the number of high voltage power supplies and control channels required by a processor in communication with the microfluidic device 5.

Once the substantially purified polypeptide enters the reaction channel 80, the substantially purified polypeptide is confined to the reaction channel 80. The present invention provides a method of performing Edman degradation on the substantially purified polypeptide while it is confined to the reaction channel.

Edman degradation has been used for sequencing proteins and peptides since its introduction by Pehr Edman. The Edman degradation method for peptide and protein sequencing is based on the cyclic removal and identification of the terminal amino acid. The Edman degradation is based on a labeling reaction between the terminal amino group and phenyl isothiocyanate, C₆H₅N═C═S. When the labeled polypeptide is treated with acid, the terminal amino acid residue splits off as an unstable intermediate that undergoes rearrangement to a phenylthiohydantoin. This last product can be identified by comparison with phenylthiohydantoin prepared from standard amino acids. If both the unstable intermediate and the phenylthiohydantoin are present, both species can be detected simultaneously, making the conversion step optional. The sequence of the substantially purified polypeptide is then elucidated by cycling the protein or peptide through many stages of removal and sequential identification of the terminal amino acid residue.

To achieve the stepwise activation of a terminal amino acid, the cleavage of the terminal amino acid, and the subsequent identification of each cleaved terminal amino acid the appropriate chemistry must be applied. Many varied chemistries have been developed for this purpose. These different methods have been developed primarily to enable different detection schemes, to improve the limits of detection, or to decrease the reaction time.

The solvent systems used for the reagents during Edman degradation often include non-polar organic solvents, such as heptane. These solvents are not amenable to electroosmotic flow and due primarily to their low degree of polarity. In a preferred embodiment, solvents that produce relatively high levels of electroosmotic flow (EOF) are used. A relatively high level of EOF is crucial to a microfluidic device that relies primarily on EOF for transport of solutions.

Each cycle of Edman degradation consists of a series of chemical reactions effected by flowing different reagents over a peptide or polypeptide which is engaged to a solid support or a peptide or polypeptide remaining in solution. Many Edman degradation chemistries are based on coupling a reagent to a terminal amino acid in order to activate the terminal amino acid and the introduction of another reagent to cleave the activated terminal amino acid. Cleaving the terminal amino acid is followed by the steps of recovering the cleaved terminal amino acid and identifying the cleaved terminal amino acid.

The coupling step of an Edman degradation reaction is typically carried out by passing a first solution of an aqueous base over the protein or polypeptide followed by passing a second solution of the coupling reagent (usually phenylisothiocynate, “PITC”) in an organic solvent over the polypeptide. Additionally, the aqueous base and the coupling reagent solutions are often cycled through 2 or 3 times to improve the coupling reaction efficiency.

Phenylisothiocyanate (PITC) is the original coupling reagent used in Edman degradation, and is still the most widely used coupling reagent. However, other coupling reagents have been developed, usually for the purpose of improved detection. A list of some of the reagents that may be used with the present invention include, but are not limited to, the following: 7-N,N-dimethylaminosulfonyl-4-(2,1,3-benzoxadiazolyl)isothiocyanate (DBD-NCS); 7-[(N,N-dimethylamino)sulfonyl]-2,1,3-benzoxadiazol-4-yl isothiocyanate (DBD-NCS); 7-[(N,N-Dimethylamino)sulfonyl]-2,1,3-benzooxadiazol-4-yl Isothiocyanate; Fluorinated isothiocyanate; Fluorescein isothiocyanate (FITC); 4-(N-1-dimethylaminonaphthalene-5-sulfonylamino)phenyl isothiocyanate (DNSAPITC); 4-N,N-dimethylaminoazobenzene 4′-isothicyanate phenyllisothiocyanate (DABITC); 2-(4-isothiocyanatophenoxy)-1,3,2-dioxaphosphinane 2-oxide (PEPITC); 3-[4′(ethylene-N,N,N-trimethylamino)phenyl]-2-isothiocyanate (P(ETAP)TH); Para-Phenylazpphenylisothiocyanate (PAPITC); and Dansyl-amino PITC.

In a preferred embodiment, the two reagent solutions are combined into one by removing water. Thus the coupling is achieved with just one solution, which is made from 5% PITC in 35% acetonitrile, 62.5 methanol and 2.55 NMM.

In other embodiments of the present invention, the following solvent systems are used: 1) 5% PITC in Heptane and 2) 25% TMA in Isopropanol/Water 1/1 v/v; 5% PITC in NMM/CAN/MeOH/Water in the ratio 2.5/12.5/35/50; 10% PITC, 10% TEA in 70% ethanol-prepared immediately before use; 5% PITC in Heptane v/v; 5% TEA in Water v/v; 5% PITC in Heptane; Methyl piperidine in n-propanol and water (25:60:15); 5% PITC in Heptane; 12.5% TMA in Water; 5% PITC in Heptane; Quadrol/TFA in water/Propanol (4:3); 5% PITC in Heptane, 5% NMM in 70/30 Methanol/Water; 5% PITC in Heptane; 12.5% TMA in water; Methanol/Water/TMA/PITC 7:1:1:1 v/v.

The basic steps in an Edman degradation comprise: (1) washing a peptide or polypeptide to prepare the peptide or polypeptide for coupling of a cleavage reagent; (2) coupling of the cleavage reagent to a terminal amino acid of the peptide or polypeptide; (3) washing the coupled terminal amino acid in preparation for cleaving the terminal amino acid of the peptide or polypeptide; (4) cleaving the terminal amino acid of the peptide or polypeptide; (5) collecting the cleaved terminal amino acid of the peptide or polypeptide; (6) the continued washing of the peptide or polypeptide which will increase the collection efficiency of the cleaved amino acid; and (7) a conversion step in which a cleaved anilothiazlinone amino acid (ATZ-AA) is converted to a more stable phenylthiohydantion (PTH-AA) by means of an addition of heat and acid. In order to achieve efficient coupling of the cleavage reagent, it is common to repeat steps (1) and (2) before proceeding to step (3). Additionally, the washing of step (3) is often repeated prior to step (4) and the washing of step (6) is often repeated before returning to step (1) to begin the next cycle of Edman degradation.

In embodiments of the present invention, the following solvent systems are used in the wash step: 1) 66% Ethyl acetate and 2) Chlorobutane; 1) Methanol and 2) Ethyl acetate/Heptane 1/1 V/V; 1) Heptane/Ethyl acetate—15:1 and 2) Heptane/Ethyl acetate—7:1; Ethyl acetate; 1) Heptane and 2) ethyl acetate and 3) acetonitrile; 1) Benzene and 2) Ethyl acetate and 3) acetonitrile; 1) methanol and 2) Heptane/ethyl acetate 1/1 v/v; 1) Heptane and 2) Ethyl acetate and 3) chlorobutane; methanol.

In one embodiment of the present invention, anhydrous TFA is used as a cleavage reagent. In one embodiment of the invention, TFA is used as a cleavage reagent. In one embodiment of the invention, HFBA is used as the cleavage reagent.

In one embodiment of the present invention, chlorobutane is used as the extraction reagent. In one embodiment, heptane/ethyl acetate (5:1) is used as the extraction reagent. In one embodiment, TFA/phosphoric acid 42.5% (9:1 v/v) is used as the extraction reagent.

In the present invention, controlling the flow of reagents to the reaction channel 80 will control the rate of the reaction. In a preferred embodiment, a centrifugal force drives the reagents to the reaction channel 80.

In a preferred embodiment of the invention, the process of Edman degradation is used to cleave an N-terminal amino acid of the substantially purified polypeptide. In a preferred embodiment, a cleavage product of the Edman degradation is the N-terminal amino acid.

In a preferred embodiment of the present invention, the process of Edman degradation is used to cleave a C-terminal amino acid of the substantially purified polypeptide. In a preferred embodiment, a cleavage product of the Edman degradation is the C-terminal amino acid.

Concentrating the cleavage product in the reaction channel 80 allows for improved detection limits and speed. In a preferred embodiment of the present invention, a cleavage product is concentrated in the reaction channel 80 before the cleavage product exits the reaction channel 80 via an exit channel 54. In a preferred embodiment, a cleavage product is concentrated in the reaction channel 80 prior to being removed from the reaction channel 80. In an embodiment of the present invention, a reaction channel 80 comprises a membrane 76 which concentrates the cleavage product before the cleavage product is removed from the reaction channel 80.

In a preferred embodiment of the present invention, a cleavage product is concentrated in front of an ultrafiltration membrane 72 or 76 in the reaction channel 80 before the cleavage product is removed from the reaction channel 80. In another embodiment of the invention, the cleavage product is concentrated on a solid extraction apparatus 78 before the cleavage product is removed from the reaction channel 80. In a preferred embodiment of the present invention, the cleavage product is electrophoretically concentrated in the reaction channel 80 of the microfluidic device 5.

In a preferred embodiment, the second ultrafiltration membrane 76 comprises pores small enough to retain peptides while allowing buffer and current to pass through. In an embodiment, the membrane comprises pores having diameters ranging from about 2 to about 30 Å. In another embodiment, the membrane is a nanofiltration membrane which has a low rejection of monovalent and divalent ions but which preferentially rejects organic compounds with a molecular weight cut off in the 200 to 500 MW range or higher (i.e., such as peptides). Nanofiltration membranes are known in the art and are available from Osmonics® (at www.osmonics.com) for example.

In an embodiment, after an appropriate period of time, flow in the reaction channel 80 is reversed and a cleavage product is delivered to a downstream separation module 14 via an exit channel 54. The amount of time necessary to carry out the above-described reactions can be optimized further by varying the reaction solution, temperature, or by vibrating the microfluidic device 5.

### Downstream Separation Module

In a currently preferred embodiment of the present invention, the microfluidic device 5 delivers a cleavage product wherein the cleavage product is a terminal amino acid cleaved from a substantially purified polypeptide traveling through a reaction channel 80 of a microfluidic device 5 to a downstream separation module 14 prior to identification. The downstream separation module can comprise one or more of the separation columns described for an upstream separation apparatus 2 above. In a preferred embodiment of the present invention, high performance liquid chromatography (“HPLC”) is used as a downstream separation apparatus 14. In a preferred embodiment, CE is used as a downstream separation apparatus 14. In a preferred embodiment, CEC is used as a downstream separation apparatus 14. In a preferred embodiment, the retention time of the cleavage product can be compared with known retention times of standard amino acids and the cleavage product can be identified.

In an embodiment, the downstream separation module 14 comprises a capillary electrophoresis apparatus including at least one separation path in communication with the microfluidic device 5 for providing a source of substantially separated cleavage products.

Capillary electrophoresis is a technique that utilizes the electrophoretic nature of molecules and/or the electroosmotic flow of samples in small capillary tubes to separate sample components. Typically a fused silica capillary of 100 μm inner diameter or less is filled with a buffer solution containing an electrolyte. Each end of the capillary is placed in a separate fluidic reservoir containing a buffer electrolyte. A potential voltage is placed in one of the buffer reservoirs and a second potential voltage is placed in the other buffer reservoir. Positively and negatively charged species will migrate in opposite directions through the capillary under the influence of the electric field established by the two potential voltages applied to the buffer reservoirs. The electroosmotic flow and the electrophoretic mobility of each component of a fluid will determine the overall migration for each fluidic component. The fluid flow profile resulting from electroosmotic flow is nearly flat. The observed mobility is the sum of the electroosmotic and electrophoretic mobilities, and the observed velocity is the sum of the electroosmotic and electrophoretic velocities.

To minimize sample loss, CE separations can be used which are capable of sample extraction. Fast CE separations in less then 1 second have been achieved, but these require extremely small injection volumes and short columns. To optimize the peak capacity and speed of a CE separation, it is necessary to determine the minimum column length for a given injection plug length (e.g., such as a sample plug). However, to maximize the peak capacity of an entire sample separation, an injection plug comprising one peak should not be mixed with peak(s) from a previous separation. If the optimized CE requires too long of a column and is too slow to avoid recombining peaks, then multiple CE separations can be run in parallel.

CE can be performed in a capillary or in a channel on the microfluidic device 5. The dimensions of CE capillary match well with the channels of a microfluidic device 5 in size. CE separations provide a more than adequate amount of sample for both MALDI-MS and ESI-MS/MS-based protein analyses (see, e.g., Feng et al., 2000, Journal of the American Society For Mass Spectrometry 11: 94-99; Koziel, New Orleans, LA 2000; Khandurina et al., 1999, Analytical Chemistry 71: 1815-1819. It should be obvious to those of skill in the art that the exact configuration of the downstream separation module 14 can be varied. In an embodiment, the downstream separation module 14 comprises a separation medium and a capillary between the ends of which an electric field is applied. The transport of a separation medium in the capillary system and the injection of the sample to be tested into the separation medium can be carried out with the aid of pumps and valves but preferably by using electric fields which are suitably applied to various points of the capillary. Analysis time can be optimized by optimizing voltages, with higher voltages between the ends of a separating path generally resulting in an increase in speed. In an embodiment of the invention, voltages of about 10-1000 V/cm are typically used resulting in separation times of about less than a few minutes.

The choice of buffers and reagents in the downstream separation module 14 are preferably optimized to be compatible with a downstream system with which it connects. Similarly, as with the upstream separation module, CE can be combined with a solid-phase extraction (SPE) CE system.

When the repulsion force of the solvated ions exceeds the surface tension of the fluid sample being electrosprayed, a volume of the fluid sample is pulled into the shape of a cone, known as a Taylor cone which extends from the tip of the capillary (see, e.g., Dole et al., 1968, Chem. Phys. 49: 2240 and Yamashita and Fenn, 1984, J. Phys. Chem. 88: 4451). The potential voltage required to initiate an electrospray is dependent on the surface tension of the solution (see, e.g., Smith, 1986, IEEE Trans. Ind Appl. IA-22: 527-535). The physical size of the capillary determines the density of electric field lines necessary to induce electrospray. The process of electrospray ionization at flow rates on the order of nanoliters per minute has been referred to as “nanoelectrospray”. However, the term “electrospray” shall be used to encompass nanospray herein.

Electroosmotic pumping is preferred for rapid delivery of a peptide mixture into a peptide analysis module 17 directly from the downstream separation module 14, especially where the peptide analysis module 17 obtains and analyzes data quickly. In an embodiment, Fast ESI-TOF machines can collect spectra at rates of 4 Hz (Liu et al., 1998, supra).

Interfacing with a MALDI apparatus is still straightforward, as automated spotters that connect capillaries and MALDI targets have been developed (see., e.g., Figeys et al., 1998, Electrophoresis 19: 2338-2347). In some instances, protein analysis time can be extended and detection limits improved by decreasing the flow rate into a peptide analysis module 17 such as an MS apparatus. As discussed above, electrospray is concentration sensitive (Kebarle et al., 1997, supra) and usually the flow rate into the MS is dictated by an upstream separation system, and is therefore not optimized for MS detection. In an embodiment, capillary HPLC-MS is operated at flow rates of about 200 nL/min (see, e.g., Gatlin et al., 1998, Analytical Biochemistry 263: 93-101) and CE-MS is operated at flow rates or about 25 nL/min. To obtain a 20-fold reduction in flow rate, the electrospray must be able to operate at flow rates of 10 nL/min for capillary HPLC-MS and at about 1 nL/min for CE-MS. Such flow rates are low, but stable electrospray has been obtained for flow rates down to 0.5 nL/min (see, e.g., Valaskovic et al., 1995, Analytical Chemistry 67: 3802-3805).

Obtaining very low flow rates (˜0.5 nL/min) at a nanospray source is more dependent on the inside diameter of the capillary than on the inside diameter of the spray tip (Valaskovic, 1995, supra). In an embodiment of the invention, a capillary with a small inside diameter (5-10 μm) is used to interface the downstream separation module to the MS system. In an embodiment, the capillary is interfaced directly with an about 50 μm reaction channel 80 on the microfluidic device 5.

In a further embodiment of the invention, microfluidic device is physically separated from a plurality of nanospray needles which can be aligned for transfer of solution subject to an operator's control (directly or through a processor), using a rotary system similar to one developed for loading microfabricated capillary arrays (see, e.g., Scherer et al., 1999, Electrophoresis 20: 1508-1517). Recently, arrays of electrospray needles have been fabricated on silicon devices(see, e.g., Zubritsky et al., 2000, Anal. Chem. 72: 22A; Licklider et al., Anal. Chem. 72: 367-375).

Each sample band stored in a channel and delivered into the peptide analysis module 17 is not necessarily pure. However, unresolved peaks are common in systems such as capillary LC-MS/MS and all must be analyzed in a very short time. One great advantage of the integrated microfluidic proteomic system 1 according to the present invention is that the nanospray interface allows adequate time to analyze unresolved peptides. Separation and/or focusing by the downstream separation module 14 is a crucial step because sample concentration can be increased by orders of magnitude through sample extraction and concentration. The extraction and concentration capabilities of the integrated system 1 allow a peptide analysis module 17 such as an MS apparatus to analyze a peptide solution of much higher concentration.

### Detectors

Detectors 23 are used for the identification of a cleavage product from a cleavage reaction. In a preferred embodiment of the present invention, an ESI-MS detector is used for the identification of a cleavage product. In a preferred embodiment, a separation combined with a spectroscopic detection is a preferred detection scheme. In an embodiment, as shown in FIG. 1, detectors 23 are placed at various flow points of the system 1 to enable a user to monitor separation efficiency. In an embodiment, one or more spectroscopic detectors 23 can be positioned in communication with various channels, outputs and/or modules of the system 1. Spectroscopic detectors rely on a change in refractive index, ultraviolet and/or visible light absorption, or fluorescence after excitation of a sample (e.g., a solution including proteins) with light of a suitable wavelength.

In another embodiment of the present invention, sample bands including substantially separated proteins (e.g., obtained after passage through the upstream separation module 2) or substantially purified polypeptides (e.g., obtained after passage through the microfluidic device 5 and the downstream separation module 14) are actively sensed by optical detectors which recognize changes in a source light (e.g., such as a ultraviolet source) reacting with the sample bands. In response to such changes the detectors 23 produce one or more electrical signals which are received and processed by processors 18 in electrical communication with the detectors 23.

In a preferred embodiment of the invention, a detector 23 is provided which detects the fluorescence of the cleaved amino acid which pass through various modules of the integrated proteomic analysis system 1. All PTH amino acids are fluorescent. Most coupling reagents, including PITC, yield products that are both absorbent and fluorescent. In another embodiment, the detector 23 comprises a laser (e.g., a 210-290 nm laser) for excitation of a sample band as it passes within range of detection optics within the system and collects spectra emitted from the polypeptides, partially digested polypeptides, or peptides within the sample band in response to this excitation. The detector 23 can comprise a lens or objectives to further focus light transmitted from the laser or received from polypeptides/peptides.

In an embodiment, the detector 23 transmits signals corresponding to the emission spectra detected to the processor 18 of the integrated system 1 and the processor 18 records the time and place (e.g., module within the system) from which the signals are obtained. Detectors 23 for detecting native fluorescence of polypeptides and peptides and which are able to spectrally differentiate at least tryptophan and tyrosine are known in the art, and described, for example in Timperman et al., 1995, Analytical Chemistry 67(19): 3421-3426, the entirety of which is incorporated by reference herein. As discussed above, the detector 23 can be used to monitor and control sample flow through the integrated proteomic analysis system 1.

In an embodiment, a detector 23 is integrated into the microfluidic device 5 within the integrated proteomic analysis system 1. In an embodiment, a UV or thermal lens detector can be used and integrated into the microfluidic module 5. Recent advancements have been made with both detection systems, and limits of detection for these systems are in the low nanomolar range (see, e.g., Culbertson et al., 1999, Journal of Microcolumn Separations 11: 652-662.) In an embodiment of the invention, a UV detection system with a multi-reflection cell is integrated into a microfluidic device 5 within the integrated proteomic analysis system 1 (see, e.g., as described in Salimi-Moosavi et al., 2000, Electrophoresis 21: 1291-1299). Extremely low yoctomole detection limits have been achieved on-device with a thermal-lens detector (see, e.g., Sato et al., 1999, Analytical Sciences 15: 525-529).

In another embodiment of the invention, as shown in FIG. 1, a detector 23 is placed in optical communication between the upstream separation module 2 and the entrance channel 52 of the microfluidic device 5. The detector 23 detects sample bands delivered by the upstream separation module 2 to the microfluidic device 5 and the processor 18 in response to the signals received from the detector 23 performs a background subtraction which eliminating background electrolyte signal as sample bands are directed to one of the reaction channels 80 in the microfluidic device 5. “Cutting” the sample bands allows the peptide analysis module 17 to spend more of its time on sample analysis and less on analysis of background electrolytes. For low concentration protein samples, a very small fraction of the time (<2%) actually is spent analyzing the sample.

In an embodiment of the present invention, the peptide analysis module 17 comprises its own detector (not shown) which detects spectral information obtained from peptides being analyzed by the system 17. In another embodiment, the protein analysis detector 23 can detect various charged forms of peptide ions as they pass through a peptide analysis module 17, such as an ESI MS/MS system.

As discussed above, in an embodiment, one or more detectors 23 (including the protein analysis detector) are electrically linked to a processor 18. As used herein, the term “linked” comprises either a direct link (e.g., a permanent or intermittent connection via a conducting cable, an infra-red communicating apparatus, or the like) or an indirect link such that data are transferred via an intermediate storage apparatus (e.g. a server or a floppy disk). It will readily be appreciated that the output of the detector 23 should be in a format that can be accepted by the processor 18.

It should be obvious to those of skill in the art that a variety of detectors 23 can be selected according to the types of samples being analyzed. For example, where fluorescently labeled polypeptides/peptides are being analyzed, a laser-induced fluorescence detection system can be used which comprises a 488 nm argon ion laser (available from Uniphase, San Jose Calif.) and focussing optics (see, e.g., as described in Manz et al., 1990, Sens. Actuators, B, B1: 249-255). Detectors 23 additionally can be coupled to cameras, appropriate filter systems, and photomultiplier tubes. The detectors 23 need not be limited to optical detectors, but can comprise any detector used for detection in liquid chromatography and capillary electrophoresis, including electrochemical, refractive index, conductivity, FT-IR, and light scattering detectors, and the like.

### Processors

In a preferred embodiment, a system processor 18 is used to control flow of the cleavage products through the integrated proteomic analysis system 1, e.g., based on data obtained from detectors 23 placed at various positions in the system 1.

The system processor 18 is in communication with one or more system components (e.g., modules, detectors 23, computer workstations and the like) which in turn may have their own processors or microprocessors. These latter types of processors/microprocessors generally comprise memory and stored programs which are dedicated to a particular function (e.g., detection of fluorescent signals in the case of a detector processor, or obtaining ionization spectra in the case of a peptide analysis module processor, or controlling voltage and current settings of selected channels on a microfluidic device 5 in the case of a power supply connected to one or more microfluidic devices 5 and are generally not directly connectable to the network.

In a preferred embodiment of the invention, the system processor 18 is in communication with at least one user apparatus including a display for displaying a user interface which can be used by a user to interface with the integrated proteomic analysis system 1 (i.e., view data, set or modify system 1 parameters, and/or input data). The at least one user apparatus can be connected to an inputting apparatus such as a keyboard and one or more navigating tools including, but are not limited to, a mouse, light pen, track ball, joystick(s) or other pointing apparatus.

The system processor 18 integrates the function of processors/microprocessors associated with various system components and is able to perform one or more functions: of data interpretation (e.g., interpreting signals from other processors/microprocessors), data production (e.g., performing one or more statistical operations on signals obtained), data storage (e.g., such as creation of a relational database), data analysis (e.g., such as search and data retrieval, and relationship determination), data transmission (e.g., transmission to processors outside the system such as servers and the like or to processors in the system), display (e.g., such as display of images or data in graphical and/or text form), and task signal generation (e.g., transmission of instructions to various system components in response to data obtained from other system components to perform certain tasks).

In an embodiment of the present invention, the system processor 18 is used to control voltage differences in the various modules and channels of the integrated proteomic analysis system 1. In a preferred embodiment of the invention, this control is used to increase the amount of time the peptide analysis module 17 actually spends analyzing sample and obtaining sequence information.

In a preferred embodiment of the invention, the system processor 18 can communicate with one or more sensors (e.g., pH sensors, temperature sensors) and/or detectors 23 in communication with the modules and channels of the integrated proteomic analysis system 1. In another embodiment of the invention, the system processor 18 can modify various system parameters (e.g., reagent flow, voltage) in response to this communication. For example, the output of a detector 23 (e.g., one or more electrical signals) can be processed by the system processor 18 which can perform one or more editing functions. Editing functions comprise, but are not limited to, removing background, representing signals as images, comparing signals and/or images from duplicate or different runs, performing statistical operations (e.g., such as ensemble averaging as described in Wilm, 1996, supra), and the like. Any of these functions can be performed automatically according to operator-determined criteria, or interactively; i.e., upon displaying an image file to a human operator, the operator can modify various editing menus as appropriate. In a preferred embodiment, editing menus, for example, in the form of drop-down menus, are displayed on the interface of a user apparatus connectable to the network and in communication with the system processor 18. Alternatively, or additionally, editing menus can be accessed by selecting one or more icons, radio buttons, and/or hyperlinks displayed on the interface of the user apparatus.

In a preferred embodiment of the invention, the processor 18 is capable of implementing a program for inferring the sequence of a protein from a plurality of cleavage products or unique peptides. Such programs are known in the art and are described in Yates et al., 1991, In Techniques in Protein Chemistry II, by Academic Press, Inc. pp. 477-485; Zhou et al., The 40th ASMS Conference on Mass Spectrometry and Allied Topics, pp. 635-636; and Zhou et al., The 40th ASMS Conference on Mass Spectrometry and Allied Topics, pp. 1396-1397, the entireties of which are incorporated herein by reference.

In an embodiment, the system processor 18 can be used to determine all possible combinations of amino acids that can sum to the measured mass of an unknown peptide being analyzed after adjusting for various factors such as water lost in forming peptide bonds, protonation, other factors that alter the measured mass of amino acids, and experimental considerations that constrain the allowed combinations of amino acids. The system processor 18 can then determine linear permutations of amino acids in the permitted combinations. Theoretical fragmentation spectra are then calculated for each permutation and these are compared with an experimental fragmentation spectrum obtained for an unknown peptide to determine the amino acid sequence of the unknown peptide.

Once an experimentally determined amino acid sequence of an isolated protein or polypeptide fragment thereof has been obtained, the system processor 18 can be used to search available protein databases or nucleic acid sequence databases to determine degree of identity between the protein identified by the integrated proteomic analysis system 1 and a sequence in the database. Such an analysis may help to characterize the function of the protein. For example, in an embodiment of the invention, conserved domains within a newly identified protein can be used to identify whether the protein is a signaling protein (e.g., the presence of seven hydrophobic transmembrane regions, an extracellular N-terminus, and a cytoplasmic C-terminus would be a hallmark for a G protein coupled receptor or a GPCR).

Where a database contains one or more partial nucleotide sequences that encode at least a portion of the protein identified by the integrated proteomic analysis system 1, such partial nucleotide sequences (or their complement) can serve as probes for cloning a nucleic acid molecule encoding the protein. If no matching nucleotide sequence can be found for the protein identified by the integrated proteomic analysis system 1 within a nucleic acid sequence database, a degenerate set of nucleotide sequences encoding the experimentally determined amino acid sequence can be generated which can be used as hybridization probes to facilitate cloning the gene that encodes the protein. Clones thereby obtained can be used to express the protein.

In an embodiment of the present invention, the system processor 18 is used to generate a proteome map for a cell. In another embodiment of the present invention, the processor 18 also generates proteome maps for the same types of cells in different disease states, for the same types of cells exposed to one or more pathogens or toxins, for the same types of cells during different developmental stages, or is used to compare different types of cells (e.g., from different types of tissues). Maps obtained for cells in a particular disease state can be compared to maps obtained from cells treated with a drug or agent and can be generated for cells at different stages of disease (e.g., for different stages or grades of cancer).

In an embodiment of the present invention, the system processor 18 is used to compare different maps obtained to identify differentially expressed polypeptides in the cells described above. In another embodiment of the invention, the processor 18 displays the results of such an analysis on the display of a user apparatus, displaying such information as polypeptide name (if known), corresponding amino acid sequence and/or gene sequence, and any expression data (e.g., from genomic analyses) or functional data known. In another embodiment, data relating to proteome analysis is stored in a database along with any clinical data available relating to patients from whom cells were obtained.

In an embodiment of the present invention, the display comprises a user interface which displays one or more hyperlinks which a user can select to access various portions of the database. In another embodiment of the invention, the processor 18 comprises or is connectable to an information management system which can link the database with other proteomic databases or genomic databases (e.g., such as protein sequence and nucleotide sequence databases).

In another embodiment of the invention, a proteome map is obtained for a cell including a disrupted cell signaling pathway gene and the map is used to identify other polypeptides differentially expressed in the cell (as compared to a cell which comprises a functional cell signaling pathway gene). Differentially expressed proteins are identified as candidate members of the same signaling pathway.

In an embodiment of the invention, the candidate signaling pathway gene is disrupted in a model system such as a knockout animal (e.g., a mouse) to identify other genes in addition to the candidate signaling pathway gene whose expression is affected by the disruption and which are likely, therefore, to be in the same pathway. Other model systems comprise, but are not limited to, cell(s)or tissue(s) comprising antisense molecules or ribozymes which prevent translation of an mRNA encoding the candidate polypeptide. Methods of generating such model systems are known in the art. By obtaining proteome maps for multiple disrupted candidate signaling polypeptides, the position of the polypeptides in a pathway can be determined (e.g., to identify whether the polypeptides are upstream or downstream of other pathway polypeptides).

### Peptide Analysis Module

The peptide analysis module 17 is preferably some form of mass spectrometer (MS) apparatus including an ionizer, an ion analyzer and a detector. Any ionizer that is capable of producing ionized peptides in the gas phase can be used, such as anionspray mass spectrometer (Bruins et al., 1987, Anal Chem. 59: 2642-2647), an electrospray mass spectrometer (Fenn et al., 1989, Science 246: 64-71), and laser desorption apparatus (including matrix-assisted desorption ionization and surfaced enhanced desorption ionization apparatus). Any appropriate ion analyzer can be used as well, including, but not limited to, quadropole mass filters, ion-traps, magnetic sectors, time-of-flight, and Fourier Transform Ion Cyclotron Resonance (FTICR). In a preferred embodiment of the invention, a tandem MS instrument such as a triple quadropole, ion-trap, quadropole-time-of flight, ion-trap-time of flight, or an FTICR is used to provide ion spectra.

In an embodiment of the invention, molecular ions (e.g., daughter ions) generated by ionization of peptides from a delivery element (e.g., such as an electrospray) are accelerated through an ion analyzer of the peptide analysis module 17 as uncharged molecules and fragments are removed. In an embodiment, the ion analyzer comprises one or more voltage sources (e.g., such as electrodes or electrode gratings) for modulating the movement of ions to a detector component of the peptide analysis module 17. Daughter ions will travel to the detector based on their mass to charge ratio (m/z) (though generally the charge of the ions will be the same). In another embodiment of the invention, the detector produces an electric signal when struck by an ion.

Timing mechanisms which integrate those signals with the scanning voltages of the ion analyzer allow the peptide analysis module 17 to report to the processor 18 when an ion strikes the detector 23. The processor 18 sorts ions according to their m/z and the detector records the frequency of each event with a particular m/z. Calibration of the peptide analysis module 17 can performed by introducing a standard into the module and adjusting system components until the standard's molecular ion and fragment ions are reported accurately. In an embodiment, the peptide analysis module 17 in conjunction with the processor 18, plots a product ion spectra which corresponds to a plot of relative abundance of ions produced versus mass to charge ratio. The detected product ions are formed by isolating and fragmenting a parent ion (that is typically the molecular mass of a peptide molecule) in the peptide analysis module 17 (e.g., a mass spectrometer).

Generally, peptides typically fragment at the amide bond between amino acid residues and peaks correspond to particular amino acids or combinations of amino acids. While there may be additional peaks (ions) present in the product ion spectra, many of these other peaks can be predicted and their presence explained by comparison with spectral data of known compounds (e.g., standards). Many different processes can be used to fragment the parent ion to form product ions, including, but not limited to, collision-induced dissociation (CID), electron capture dissociation, and post-source decay.

Analysis of product ion spectra will vary depending upon the particular type of peptide analysis module 17 used.

For high throughput identification of polypeptides, matrix assisted laser-desorption ionization mass spectrometry (MS) peptide finger printing is the method of choice. Although this method is fast, it requires protein database matching and provides the least detailed information. When more detail is needed, ionization tandem mass spectrometry (ESI-MS/MS) is the method of choice (see, e.g., Karger et al., 1993, Anal Chem. 65: 900-906). MS/MS is capable of giving amino acid level sequence information and is required for de novo sequencing and analysis of post-translational modifications. The development of automated database searching programs to directly correlate MS/MS spectra with sequences in protein and nucleic acid databases has greatly increased throughput. New hybrid instruments are being developed to combine MALDI with MS/MS are being developed to combine MALDI with MS/MS to combine speed of analysis with amino acid sequence information. It should be apparent to those of skill in the art that as MS tools evolve new interfaces can be developed to couple microfluidic devices according to the invention with either MALDI or HIS sources.

In an embodiment, the spectra obtained by the peptide analysis module 17 are searched directly against a database for identification of the polypeptide from which the peptide originated. In another embodiment, the peptide analysis module 17 obtains sequence information directly from spectra obtained by the peptide analysis module 17 without the use of a protein or genomic database. This is especially desirable when the protein to be identified is not in a protein database. In an embodiment, rather than performing a search function to compare peptide sequences to a protein database, the processor 18 implements an algorithm for automated data analysis of spectra obtained from the peptide analysis module 17.

In an embodiment, the peptide analysis module 17 facilitates this interaction by isolating daughter ions (MS² ions) obtained from parent ions sprayed into the module (e.g., via an electrospray) and further isolating and fragmenting these to obtain granddaughter ions (MS³ ions) to thereby obtain MS³ spectra. For these types of analyses, ion-trapping instruments such as Fourier transform ion cyclotron resonance mass spectrometers and ion trap mass spectrometers are preferred.

MS³ spectra generally comprise two classes of ions: ions with the same terminus as daughter ions (MS² ions) and ions derived from internal fragments of peptides (some of this latter class comprise C-terminal residues). By identifying peaks that are common to both MS² and MS³ spectra (e.g., contained with an intersection spectrum), a partial sequence of the peptide can be read directly from the intersection spectrum based on the differences in mass of the major remaining ions. Obtaining MS³ spectra of many daughter ions of a peptide will generate many intersection spectra which in turn will generate many partial sequences of different areas of a peptide. Partial sequences can be combined to obtain the complete sequence of the peptide by correlating experimentally acquired spectra with theoretical spectra which are predicted for all of the sequences in a database. A fast Fourier transform can be used to determine the quality of the match. In an embodiment of the invention, detection limits are improved further by ensemble averaging of many spectra (Wilm, 1996, Analytical Chemistry 68: 1-8).

The speed of protein analysis will depend mainly on the voltage used to mobilize the samples, and the number of scans used by the protein analysis system for acquisition of data relating to a sample band. The number of scans can be optimized using methods routine in the art. In an embodiment, for ensemble averaging, the increase in signal-to-noise ratio is equal to the square root of the number of scans averaged, so at larger numbers of scans, there will be diminishing returns. Since increasing the number of scans will also increase analysis time, there will be an optimum number of scans to average. This number will be determined by the efficiency at which the system can load the samples into the electrospray/nanospray capillary and the complexity of the sample.

Higher concentration samples will contain more detectable peaks and will require less averaging. Because lower concentration samples will contain fewer peaks, there will be more time to acquire scans. An optical detection system, such as the one described above, can be used to measure the complexity of a sample before it reaches the MS and this information can be used to determine the optimum scan number.

The peptide analysis module 17 preferably compares the results of multiple runs of sample through the system 1. In an embodiment of the invention, the results of one run are compared to the results of another run utilizing the same protein or peptide sample. In another embodiment, the protein analysis module 17 compares multiple runs of sample which have been exposed for various periods of time to proteases within the microfluidic device 5 enabling analysis of undigested, partially digested, and completely digested proteins or polypeptides in the sample.

In another embodiment of the invention, the peptide analysis module 17 identifies post-translational modifications in cellular proteins. Generally, post-translational modifications may be classified into four groups, depending upon the site of chemical modification of the protein. In an embodiment, protein modifications may involve the carboxylic acid group of the carboxyl terminal amino acid residue, the amino group of the amino terminal amino acid residue, the side chain of individual amino acid residues in the polypeptide chain, and/or the peptide bonds in the polypeptide chain. The modifications may be further sub-grouped according to distinct types of chemical modifications, such as phosphorylation, glycosylation, acylation, amidation and carboxylation. Using MS, peptide ions are fragmented into peptide fragment ions which are selected and further fragmented to yield information relating to the nature and site of a modification.

### Uses of Cell Signaling Polypeptides

The expression and/or form (e.g., presence or absence of modifications and/or cleavage products or other processed forms) of candidate signaling pathway polypeptides can be evaluated in a plurality of biological samples to evaluate the use of these polypeptides as diagnostic molecules. The expression and/or form (e.g., sequence) of nucleic acid molecules encoding the polypeptides also can be evaluated in the plurality of biological samples as these also may be diagnostic. In an embodiment of the present invention, the biological samples are from patients having a disease (or a particular stage of a disease) or who are at risk of developing a disease. In another embodiment, the disease is a pathology involving abnormal cell proliferation or cell death (e.g., such as cancer).

When disruption of a candidate signaling pathway polypeptide (e.g., loss of expression, reduced expression, overexpression, ectopic expression of the polypeptide, or the presence of an aberrant form of the polypeptide) is identified as diagnostic of a particular disease or trait, molecular probes reactive with disrupted polypeptide can be contacted with a test sample from a patient suspected of having a disease or trait and reactivity of the molecular probe with the disrupted polypeptide can be determined as a means of determining the presence or absence or risk of having the disease or trait.

In an embodiment of the present invention, the molecular probe is reactive with both the disrupted and non-disrupted polypeptide and the presence of a disrupted polypeptide can be determined by detecting differences in molecular mass or sequence between the disrupted and non-disrupted polypeptide or detecting changes in the quantitative level of a single species of polypeptide (i.e., where the disruption changes the expression rather than the structure of the non-disrupted polypeptide). In another embodiment of the invention, the molecular probe is specifically reactive with a disrupted form of a polypeptide and does not react with a non-disrupted form of the polypeptide (e.g., the probe reacts with a phosphorylated form of a polypeptide but does not react with a non-phosphorylated form).

In an embodiment of the present invention, the probe is an antibody. Polyclonal antisera or monoclonal antibodies can be made using methods known in the art. A mammal such as a mouse, hamster, or rabbit, can be immunized with an immunogenic form of a signaling polypeptide, fragment, modified form thereof, or variant form thereof. Techniques for conferring immunogenicity on such molecules comprise conjugation to carriers or other techniques well known in the art. In an embodiment, the immunogenic molecule can be administered in the presence of adjuvant. Immunization can be monitored by detection of antibody titers in plasma or serum. Standard immunoassay procedures can be used with the immunogen as antigen to assess the levels and the specificity of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (see, e.g., Kohler and Milstein, 1975, Nature 256: 495-497; Kozbor et al., 1983, Immunol. Today 4: 72, Cole et al., 1985, In Monoclonal Antibodies in Cancer Therapy, Allen R. Bliss, Inc., pages 77-96). Additionally, techniques described for the production of single-chain antibodies (U.S. Pat. No. 4,946,778) can be adapted to produce antibodies according to the invention.

Antibody fragments which contain specifically bind to a cell signaling polypeptide, modified forms thereof, and variants thereof, also may be generated by known techniques. In an embodiment, such fragments comprise, but are not limited to, F(ab′)₂ fragments which can be produced by pepsin digestion of the antibody molecule and the Fab fragments which can be generated by reducing the disulfide bridges of the F(ab′)₂ fragments. VH regions and FV regions can be expressed in bacteria using phage expression libraries (e.g., Ward et al., 1989, Nature 341: 544-546; Huse et al., 1989, Science 246: 1275-1281; McCafferty et al., 1990, Nature 348: 552-554).

Chimeric antibodies, i.e., antibody molecules that combine a non-human animal variable region and a human constant region also are within the scope of the invention. Chimeric antibody molecules comprise, for example, the antigen binding domain from an antibody of a mouse, rat, or other species, with human constant regions. Standard methods may be used to make chimeric antibodies containing the immunoglobulin variable region which recognizes the gene product of cell signaling polypeptides (see, e.g., Morrison et al., 1985, Proc. Natl. Acad. Sci. USA 81: 6851; Takeda et al., 1985, Nature 314: 452; U.S. Pat. No. 4,816,567; U.S. Pat. No. 4,816,397). Chimeric antibodies are preferred where the probes are to be used therapeutically to treat a condition associated with physiological responses to an aberrant cell signaling pathways.

Monoclonal or chimeric antibodies can be humanized further by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (see, e.g., Teng et al., 1983, Proc. Natl. Acad. Sci. USA 80: 7308-7312; Kozbor et al., 1983, Immunology Today 4: 7279; Olsson et al., 1982, Meth. Enzymol. 92: 3-16; WO 92/06193; EP 0239400).

In an embodiment of the present invention, an antibody is provided which recognizes a modified and/or variant form of an cell signaling polypeptide but which does not recognize a non-modified and/or non-variant form of the cell signaling polypeptide. In an embodiment, peptides including the variant region of a variant polypeptide can be used as antigens to screen for antibodies specific for these variants. Similarly modified peptides or proteins can be used as immunogens to select antibodies which bind only to the modified form of the protein and not to the unmodified form. Methods of making variant-specific antibodies and modification-specific antibodies are known in the art and described in U.S. Pat. No. 6,054,273; U.S. Pat. No. 6,054,273; U.S. Pat. No. 6,037,135; U.S. Pat. No. 6,022,683; U.S. Pat. No. 5,702,890; U.S. Pat. No. 5,702,890, and in Sutton et al., 1987, J. Immunogenet 14(1): 43-57, for example, the entireties of which are incorporated by reference herein.

In an embodiment of the present invention, labeled antibodies or antigen-binding portions thereof are provided. Antibodies can be labeled with a fluorescent compound such as fluorescein, amino coumarin acetic acid, tetramethylrhodamine isothiocyanate (TRITC), Texas Red, Cy3.0 and Cy5.0. GFP is also useful for fluorescent labeling, and can be used to label antibodies or antigen-binding portions thereof by expression as fusion proteins. GFP-encoding vectors designed for the creation of fusion proteins are commercially available. Other labels comprise, but are not limited to, alkaline phosphatase, beta-galactosidase, or acetylcholinesterase; luminescent materials such as luminol; radioactive materials, electron dense substances, such as ferritin or colloidal gold, and other molecules such as biotin.

Polypeptides and/or modified forms thereof and/or variants thereof can be detected using standard immunoassays using the antibodies described above. Immunoassays comprise, but are not limited to, radioimmunoassays, enzyme immunoassays (e.g. ELISA), immunofluorescence (such as immunohistochemical analyses), immunoprecipitation, latex agglutination, hemagglutination, and histochemical tests. Such assays are routine in the art.

In another embodiment of the present invention, a plurality of different probes are stably associated at different known locations on a solid support. In a still more preferred embodiment of the invention, the different probes represent different signaling polypeptides in the same signaling pathway. In an embodiment of the invention, at least one early pathway probe (i.e., reactive with at least one early pathway polypeptide, downstream of fewer than about 5 pathway polypeptides) and at least one late pathway probe (i.e., reactive with at least one late pathway polypeptide, downstream of greater than about 10 pathway polypeptides). In another embodiment of the invention, at least about one middle pathway probe is provided (i.e., reactive with at least one middle pathway polypeptide, downstream of greater than about five but less about 10 pathway polypeptides). In another embodiment of the invention, one or more reactions control polypeptides reactive with a constitutively expressed polypeptide (e.g., actin) is provided. One or more background control probes (e.g., reactive with a polypeptide not expected to be in a particular sample, such as a probe reactive with a plant polypeptide where a human sample is evaluated) also is provided. The support and probes can be reacted with a biological sample comprising polypeptides from cell(s) or tissue(s) of a patient (which are preferably labeled) and used to identify cell signaling polypeptides or modified or variant forms thereof expressed in the sample by determining which of the probes on the support react with cellular polypeptides in the sample.

It should be obvious to those of skill in the art that parallel assays can be performed with molecular probes reactive with nucleic acids encoding the cell signaling polypeptides according to the invention. Hybridization-based assays such as Southerns (e.g., to detect deleted or other mutated cell signaling genes), Northerns, RT-PCR, array-based assays and the like (e.g., to detect altered expression of transcripts or the expression of aberrant transcripts corresponding to cell signaling genes identified according to methods of the invention). Such assays are routine in the art.

Cells genetically engineered to express recombinant cell signaling polypeptides according to the invention can be used in a screening program to identify other cellular biomolecules or drugs that specifically interact with the recombinant protein, or to produce large quantities of the recombinant protein, e.g., for therapeutic administration. Possession of cloned genes encoding the cell signaling polypeptides according to the invention permits gene therapy to replace or supplement such polypeptides where the absence or diminished expression of the polypeptides is associated with disease.

Variations, modification, and other implementations of what is described herein will occur to those of skill in the art without departing from the spirit and scope of the invention and the following claims. All references, patents and patent publications cited herein are hereby incorporated by reference in their entireties.

What is claimed is:

1. A microfluidic device comprising:
a substrate defining an entrance channel;
at least one reaction channel engaging the entrance channel;
at least one reagent reservoir engaging the reaction channel, the reagent reservoir being capable of delivering at least one reagent to the reaction channel;
the at least one reagent necessary for cleaving a terminal amino acid from a substantially purified polypeptide; and
an exit channel extending from the reaction channel.

2. The device of claim 1 wherein the substantially purified polypeptide is cleaved from the C-terminal end of the substantially purified polypeptide.

3. The device of claim 1 wherein a solid support engages the substantially purified polypeptide in the reaction channel to confine the substantially purified polypeptide to the reaction channel.

4. The device of claim 3 wherein the solid support is a membrane.

5. The device of claim 3 wherein the solid support is a plurality of beads.

6. The device of claim 5 wherein the plurality of beads are magnetic.

7. The device of claim 6 wherein an external force confines the plurality of magnetic beads to the reaction channel.

8. The device of claim 5 wherein a blocking structure confines the plurality of beads to the reaction channel by blocking the plurality of beads from exiting the reaction channel.

9. The device of claim 7 further comprising a blocking structure which blocks the plurality of beads from exiting the reaction channel.

10. The device of claim 8 wherein the blocking structure blocks the plurality of beads but does not impede the flow of the cleavage product.

11. The device of claim 8 wherein the blocking structure blocks the plurality of beads and impedes the flow of the cleavage product.

12. The device of claim 11 wherein the blocking structure is an ultrafiltration membrane.

13. The device of claim 1 wherein an at least one ultrafiltration membrane confines the substantially purified polypeptide to the reaction channel.

14. The device of claim 5 wherein the plurality of beads can be placed into and removed from the reaction channel by using bead injection.

15. The device of claim 1 further comprising a plurality of reagent reservoirs, each reagent reservoir engaging at least one reaction channel and the plurality of reagent reservoirs used for delivering a plurality of reagents to at least one reaction channel.

16. The device of claim 1 wherein the microfluidic device is approximately circular in shape.

17. The device of claim 1 wherein the reagent is forced from the reagent reservoir to the reaction channel by a centrifugal movement of the microfluidic device.

18. The device of claim 1 wherein the reagent is forced from the reagent reservoir to the reaction channel by a hydrodynamic pumping.

19. The device of claim 1 wherein the reagent is forced from the reagent reservoir to the reaction channel by an electrokinetic pumping.

20. A microfluidic system for proteome analysis comprising:
an upstream separation module for producing a substantially purified polypeptide; and
a microfluidic device engaging the upstream separation module, wherein the substantially purified polypeptide undergoes a cleavage reaction on the microfluidic device, producing a cleavage product.

21. The device of claim 20 further comprising a downstream separation module engaging the microfluidic device for separating a cleavage product from a by-product of the cleavage reaction.

22. The system of claim 20 wherein the upstream separation module separates a plurality of polypeptides according to an at least first criteria.

23. The system of claim 20 wherein the upstream separation module produces a substantially purified polypeptide by separating a plurality of peptides according to a first criteria and a second criteria.

24. The system of claim 20 further comprising a first separation path for separating the plurality of polypeptides according to the first criteria and a second separation path for separating the plurality of polypeptides according to the second criteria.

25. The system of claim 20 wherein the substantially purified polypeptide undergoes a process of Edman degradation to produce a cleavage product.

26. The system of claim 21 wherein the downstream separation module is a liquid chromatographic column.

27. The system of claim 21 wherein the downstream separation module is a capillary electrophoresis column.

28. The system of claim 21 wherein the downstream separation module is a capillary electrochromatography device.

29. The system of claim 21 wherein a detector is used for the detection of the cleavage product following separation of the cleavage product from the by-product of the cleavage reaction.

30. The system of claim 29 wherein the detector is in communication with a processor for identifying an amino acid sequence of the cleavage product.

31. The system of claim 21 wherein the downstream separation module is in communication with a peptide analysis module.

32. The system of claim 30 wherein an information related to the amino acid sequence is stored in a database.

33. A method of protein digestion on a microfluidic device comprising:
(a) delivering a substantially purified polypeptide to a reaction channel;
(b) confining the substantially purified polypeptide in the reaction channel;
(c) digesting the substantially purified polypeptide in the reaction channel producing a cleavage product; and
(d) removing the cleavage product from the reaction channel
wherein steps (c) and (d) are repeated until the substantially purified polypeptide has been substantially digested.

34. The method of claim 33 wherein Edman degradation is used to digest the substantially purified polypeptide.

35. The method of claim 33 wherein the substantially purified polypeptide is confined in the reaction channel by immobilizing the substantially purified polypeptide on a solid support capable of engaging the substantially purified polypeptide.

36. The method of claim 35 wherein the solid support engages the substantially purified polypeptide at a C-terminal end of the substantially purified polypeptide.

37. The method of claim 36 wherein a single amino acid is cleaved from an N-terminal end of the substantially purified polypeptide and the cleaved single amino acid is the cleavage product.

38. The method of claim 35 wherein the solid support is a plurality of beads.

39. The method of claim 38 wherein the plurality of beads are magnetic.

40. The method of claim 39 wherein the plurality of magnetic beads are confined in the reaction channel by application of an external force.

41. The method of claim 38 wherein the plurality of beads are confined in the reaction channel by a blocking structure which blocks the plurality of beads from exiting the reaction channel.

42. The method of claim 40 further comprising a blocking structure which blocks the plurality of beads from exiting the reaction channel.

43. The method of claim 41 wherein the blocking structure blocks the plurality of beads but does not impede the flow of the cleavage product.

44. The method of claim 41 wherein the blocking structure blocks the plurality of beads and impedes the flow of the cleavage product.

45. The method of claim 44 wherein the blocking structure is an ultrafiltration membrane.

46. The method of claim 35 wherein the solid support is a membrane.

47. The method of claim 33 wherein the substantially purified polypeptide is confined in the reaction channel using an ultrafiltration membrane.

48. The method of claim 38 wherein the plurality of beads can be placed into and removed from the reaction channel by using bead injection.

49. The method of claim 33 wherein the microfluidic device is circular in shape.

50. The method of claim 33 further comprising adding a reagent to the reaction channel.

51. The method of claim 33 further comprising adding a plurality of reagents to the reaction channel.

52. The method of claim 50 wherein the reagent is forced from a reagent reservoir to the reaction channel by a centrifugal movement of the microfluidic device.

53. The method of claim 33 wherein the cleavage product is concentrated before exiting the reaction channel.

54. The method of claim 33 wherein the solid support engages the substantially purified polypeptide at a N-terminal end of the substantially purified polypeptide.

55. The method of claim 54 wherein a single amino acid is cleaved from a C-terminal end of the substantially purified polypeptide and the cleaved single amino acid is the digestion product.

56. A method for proteome analysis comprising:
(a) delivering a substantially purified polypeptide from an upstream separation module to a microfluidic device;
(b) digesting the substantially purified polypeptide on a microfluidic device to produce a digestion product;
(c) separating the multiple digestion products from each other in a downstream separation module; and
(d) digesting the separated digestion products by Edman degradation.

57. The method of claim 56 wherein the upstream separation module produces a substantially purified polypeptide by separating a plurality of peptides according to a first criteria and a second criteria.

58. The method of claim 57 further comprising a first separation path for separating the plurality of polypeptides according to the first criteria.

59. The method of claim 58 further comprising a second separation path for separating the plurality of polypeptides according to a second criteria.

60. The method of claim 56 wherein the process of Edman degradation digests the substantially purified polypeptide and produces a cleavage product.

61. The method of claim 56 wherein the downstream separation module is a liquid chromatography column.

62. The method of claim 56 wherein the downstream separation module is a capillary electrophoresis column.

63. The method of claim 56 wherein the downstream separation module is a capillary eletrochromatography device.

64. The method of claim 56 wherein a fluorescence detector is used for the detection of the digestion product.

65. The method of claim 56 wherein the downstream separation module is in communication with a peptide analysis module.

66. The method of claim 65 wherein a processor is in communication with the peptide analysis module for determining a amino acid sequence of the digestion product.

67. The method of claim 66 further comprising the processor in communication with the peptide analysis module for determining a amino acid sequence of the substantially purified polypeptide.

68. The method of claim 67 wherein the information related to the amino acid sequence is stored in a database.
